SEQUENCE LISTING (64 sequences)

SEQ ID NO: 1 - protein, 1532 aa, Homo sapiens
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 2 - protein, 1515 aa, Homo sapiens
MSLLTCAFYLGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 3 - protein, 1516 aa, Homo sapiens
MAPILSINLFIGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 4 - protein, 1532 aa, synthetic construct
  REGION 1..1532: note = GDE Human Isoform 1 variant
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 5 - protein, 1515 aa, synthetic construct
  REGION 1..1515: note = GDE Human Isoform 5 variant
MSLLTCAFYLGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 6 - protein, 1516 aa, synthetic construct
  REGION 1..1516: note = GDE Human Isoform 6 variant
MAPILSINLFIGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVTRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLAYRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 7 - protein, 6 aa, synthetic construct
  REGION 1..6: note = N-ter amino acids of d1b4
MGSFQY

SEQ ID NO: 8 - protein, 6 aa, synthetic construct
  REGION 1..6: note = N-ter amino acids of d1b5
MEKSGG

SEQ ID NO: 9 - protein, 6 aa, synthetic construct
  REGION 1..6: note = N-ter amino acids of d1b6
MILRVG

SEQ ID NO: 10 - protein, 6 aa, synthetic construct
  REGION 1..6: note = N-ter amino acids of d1b7
MGADNH

SEQ ID NO: 11 - protein, 6 aa, synthetic construct
  REGION 1..6: note = N-ter amino acids of d1b8
MLDCVT

SEQ ID NO: 12 - protein, 1445 aa, synthetic construct
  REGION 1..1445: note = d1b4
MGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 13 - protein, 1434 aa, synthetic construct
  REGION 1..1434: note = d1b5
MEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 14 - protein, 1422 aa, synthetic construct
  REGION 1..1422: note = d1b6
MILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 15 - protein, 1418 aa, synthetic construct
  REGION 1..1418: note = d1b7
MGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 16 - protein, 1410 aa, synthetic construct
  REGION 1..1410: note = d1b8
MLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 17 - protein, 193 aa, synthetic construct
  REGION 1..193: note = N terminal part of d1b4
MGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHH

SEQ ID NO: 18 - protein, 182 aa, synthetic construct
  REGION 1..182: note = N terminal part of d1b5
MEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHH

SEQ ID NO: 19 - protein, 170 aa, synthetic construct
  REGION 1..170: note = N terminal part of d1b6
MILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHH

SEQ ID NO: 20 - protein, 166 aa, synthetic construct
  REGION 1..166: note = N terminal part of d1b7
MGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHH

SEQ ID NO: 21 - protein, 158 aa, synthetic construct
  REGION 1..158: note = N terminal part of d1b8
MLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHH

SEQ ID NO: 22 - DNA, 4338 bp, synthetic construct
  misc_feature 1..4338: note = DNA sequence d1b4 (codon-optimized)
  source 1..4338: mol_type = other DNA
atgggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtga

SEQ ID NO: 23 - DNA, 4338 bp, synthetic construct
  misc_feature 1..4338: note = DNA sequence d1b4 (non optimized)
  source 1..4338: mol_type = other DNA
atgggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatag

SEQ ID NO: 24 - DNA, 4305 bp, synthetic construct
  misc_feature 1..4305: note = DNA sequence d1b5 (codon-optimized)
  source 1..4305: mol_type = other DNA
atggagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtga

SEQ ID NO: 25 - DNA, 4305 bp, synthetic construct
  misc_feature 1..4305: note = DNA sequence d1b5 (non optimized)
  source 1..4305: mol_type = other DNA
atggagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatag

SEQ ID NO: 26 - DNA, 4269 bp, synthetic construct
  misc_feature 1..4269: note = DNA sequence d1b6 (codon-optimized)
  source 1..4269: mol_type = other DNA
atgattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtga

SEQ ID NO: 27 - DNA, 4269 bp, synthetic construct
  misc_feature 1..4269: note = DNA sequence d1b6 (non-optimized)
  source 1..4269: mol_type = other DNA
atgattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatag

SEQ ID NO: 28 - DNA, 4257 bp, synthetic construct
  misc_feature 1..4257: note = DNA sequence d1b7 (codon-optimized)
  source 1..4257: mol_type = other DNA
atgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtga

SEQ ID NO: 29 - DNA, 4257 bp, synthetic construct
  misc_feature 1..4257: note = DNA sequence d1b7 (non optimized)
  source 1..4257: mol_type = other DNA
atgggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatag

SEQ ID NO: 30 - DNA, 4233 bp, synthetic construct
  misc_feature 1..4233: note = DNA sequence d1b8 (codon-optimized)
  source 1..4233: mol_type = other DNA
atgctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtga

SEQ ID NO: 31 - DNA, 4233 bp, synthetic construct
  misc_feature 1..4233: note = DNA sequence d1b8 (non optimized)
  source 1..4233: mol_type = other DNA
atgttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatag

SEQ ID NO: 32 - DNA, 4599 bp, Homo sapiens
  source 1..4599: mol_type = other DNA
atgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatag

SEQ ID NO: 33 - DNA, 4599 bp, synthetic construct
  misc_feature 1..4599: note = Optimized Nucleic acid sequence encoding full size WT GDE isoform 1
  source 1..4599: mol_type = other DNA
atgggccacagcaagcagatcagaatcctgctgctgaacgagatggaaaagctggaaaagaccctgttccggctcgagcagggctacgagctgcagtttagactgggccctacactgcagggcaaagccgtgaccgtgtacacaaactaccccttccctggcgaaaccttcaaccgcgagaagttcagaagcctggactgggagaaccccaccgagagagaggacgacagcgacaagtactgcaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtga

SEQ ID NO: 34 - DNA, 441 bp, synthetic construct
  misc_feature 1..441: note = HBB2 intron
  source 1..441: mol_type = other DNA
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 35 - DNA, 441 bp, synthetic construct
  misc_feature 1..441: note = modified HBB2 intron
  source 1..441: mol_type = other DNA
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 36 - DNA, 1438 bp, synthetic construct
  misc_feature 1..1438: note = FIX intron
  source 1..1438: mol_type = other DNA
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctatgtcaaacatcttggagttgatatttggggaaacacaatactcagttgagttccctaggggagaaaagcacgcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttca

SEQ ID NO: 37 - DNA, 1438 bp, synthetic construct
  misc_feature 1..1438: note = Modified FIX intron
  source 1..1438: mol_type = other DNA
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacattgcttttgttgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaattgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctttgtcaaacatcttggagttgatatttggggaaacacaatactcagttgagttccctaggggagaaaagcacgcttaagaattgacataaagagtaggaagttagctattgcaacatatatcactttgttttttcacaactacagtgactttttgtatttcccagaggaaggcatacagggaagaaattatcccatttggacaaacagcttgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgttggatcactttgcaaagatcctcattgagctattttcaagtgttgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcttgttgttttcttttttgctaaaactaaagaattattcttttacatttca

SEQ ID NO: 38 - DNA, 881 bp, synthetic construct
  misc_feature 1..881: note = Chicken beta-globin intron
  source 1..881: mol_type = other DNA
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctc

SEQ ID NO: 39 - DNA, 881 bp, synthetic construct
  misc_feature 1..881: note = modified chicken-beta globin intron
  source 1..881: mol_type = other DNA
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttttggtaatcgtgcgagagggcgcagggacttcctttgtcccaaatctgtgcggagccgaaatctgggaggcgccgccgcaccccctctagcgggcgcggggcgaagcggtgcggcgccggcaggaaggaattgggcggggagggccttcgtgcgtcgccgcgccgccgtccccttctccctc

SEQ ID NO: 40 - DNA, 17 bp, synthetic construct
  misc_feature 1..17: note = sNRP1 polyadenylation signal
  source 1..17: mol_type = other DNA
aaataaaatacgaaatg

SEQ ID NO: 41 - DNA, 169 bp, synthetic construct
  misc_feature 1..169: note = bGH polyadenylation signal
  source 1..169: mol_type = other DNA
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 42 - DNA, 58 bp, synthetic construct
  misc_feature 1..58: note = pA58 polyadenylation signal
  source 1..58: mol_type = other DNA
gcggccgcaataaaagatctttattttcattagatctgtgtgttggttttttgtgtgt

SEQ ID NO: 43 - DNA, 351 bp, synthetic construct
  misc_feature 1..351: note = mini CMV promoter
  source 1..351: mol_type = other DNA
gcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 44 - DNA, 4769 bp, synthetic construct
  misc_feature 1..4769: note = Cassette d1b4
  source 1..4769: mol_type = other DNA
gcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgttaattaagccaccatgggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtgagtttaaacgcggccgcaataaaagatctttattttcattagatctgtgtgttggttttttgtgtgt

SEQ ID NO: 45 - DNA, 4736 bp, synthetic construct
  misc_feature 1..4736: note = Cassette d1b5
  source 1..4736: mol_type = other DNA
gcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgttaattaagccaccatggagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtgagtttaaacgcggccgcaataaaagatctttattttcattagatctgtgtgttggttttttgtgtgt

SEQ ID NO: 46 - DNA, 4700 bp, synthetic construct
  misc_feature 1..4700: note = Cassette d1b6
  source 1..4700: mol_type = other DNA
gcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgttaattaagccaccatgattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtgagtttaaacgcggccgcaataaaagatctttattttcattagatctgtgtgttggttttttgtgtgt

SEQ ID NO: 47 - DNA, 4688 bp, synthetic construct
  misc_feature 1..4688: note = Cassette d1b7
  source 1..4688: mol_type = other DNA
gcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgttaattaagccaccatgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtgagtttaaacgcggccgcaataaaagatctttattttcattagatctgtgtgttggttttttgtgtgt

SEQ ID NO: 48 - DNA, 4664 bp, synthetic construct
  misc_feature 1..4664: note = Cassette d1b8
  source 1..4664: mol_type = other DNA
gcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgttaattaagccaccatgctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtgagtttaaacgcggccgcaataaaagatctttattttcattagatctgtgtgttggttttttgtgtgt

SEQ ID NO: 49 - DNA, 4722 bp, synthetic construct
  misc_feature 1..4722: note = Cassette d1b3
  source 1..4722: mol_type = other DNA
gcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgagatctgccaccatgggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtgagtttaaacgcggccgcaataaaagatctttattttcattagatctgtgtgttggttttttgtgtgt

SEQ ID NO: 50 - DNA, 5028 bp, synthetic construct
  misc_feature 1..5028: note = Cassette GDE FS
  source 1..5028: mol_type = other DNA
gcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgagatctgccaccatgggccacagcaagcagatcagaatcctgctgctgaacgagatggaaaagctggaaaagaccctgttccggctcgagcagggctacgagctgcagtttagactgggccctacactgcagggcaaagccgtgaccgtgtacacaaactaccccttccctggcgaaaccttcaaccgcgagaagttcagaagcctggactgggagaaccccaccgagagagaggacgacagcgacaagtactgcaagctgaacctgcagcagagcggctccttccagtactacttcctgcaaggcaacgagaagtccggcggaggctacatcgtggtggaccctattctgagagtgggcgccgacaatcacgtgctgcctctggattgtgtgaccctgcagaccttcctggccaagtgtctgggccctttcgatgagtgggagagcagactgcgcgtggccaaagaaagcggctacaacatgatccacttcacccctctgcagaccctgggcctgagcagaagctgttacagcctggccaaccagctggaactgaaccccgacttcagcagacccaaccggaagtacacctggaacgatgtgggccagctggtggaaaaactgaagaaagaatggaacgtgatctgcatcaccgacgtggtgtacaaccacaccgccgccaacagcaagtggatccaagagcaccctgagtgcgcctacaacctggtcaacagccctcacctgaaacctgcctgggtgctcgatagagccctgtggcggtttagctgtgatgtggccgagggcaagtacaaagagaagggcatccccgctctgatcgagaacgaccaccacatgaacagcatccggaagatcatctgggaagatattttccccaagctgaagctgtgggagttcttccaggtggacgtgaacaaggccgtggaacagttcagacggctgctgacccaagagaacagaagagtgaccaagagcgaccccaaccagcacctgaccatcattcaggaccccgagtatcggagattcggctgcaccgtggacatgaatatcgccctgaccaccttcattccccacgacaaaggacctgccgccatcgaggaatgctgcaactggttccacaagcggatggaagaattgaacagcgagaagcaccggctgatcaactaccaccaagagcaggccgtgaactgcctgctgggcaacgtgttctatgagagactggccggacacggccctaagctgggacctgtgacaagaaagcaccctctggttacccggtacttcacctttccattcgaagagatcgacttctccatggaagagagcatgatccatctgcctaacaaggcctgcttcctgatggctcacaacggctgggttatgggcgacgaccctctgagaaatttcgccgagcctggcagcgaggtgtacctgagaagagaactgatctgttggggcgacagcgtgaagctgagatacggcaacaagcccgaggactgcccttacctgtgggcccatatgaagaagtacacagagatcaccgccacctactttcagggcgtcagactggacaactgccacagcacacctctgcacgtggccgagtacatgctggacgccgctagaaatctgcagcccaacctgtatgtggtggccgagctgtttaccggctccgaggacctggacaatgtgttcgtgaccagactgggcatcagcagcctgatcagagaagccatgtccgcctacaatagccacgaagagggcagactggtgtacagatatggcggcgagcctgtgggcagcttcgttcagccttgtctgaggcctctgatgcccgccattgctcacgccctgttcatggacatcacccacgataacgagtgccccatcgtgcacagaagcgcctacgacgctctgcctagcaccaccattgtgtccatggcctgttgtgccagcggcagcacaagaggctatgacgaactggtgccccaccagatttccgtggtgtccgaggaacggttctacaccaagtggaaccccgaggctctgcccagcaataccggcgaagtgaatttccagagcggcatcattgccgccagatgcgccatcagcaagctgcaccaagaactgggcgccaagggcttcattcaggtgtacgtggaccaggtcgacgaggacattgtggccgtgacaagacacagccccagcatccatcagagcgtggtggctgtgagcagaaccgccttcagaaaccccaagaccagcttctacagcaaagaggtgccccagatgtgcatccccggcaagattgaggaagtggtgctcgaggcccggaccatcgagagaaacaccaagccttaccggaaggacgagaactccatcaacggcacccctgacatcaccgtggaaatcagagagcacatccagctcaacgagagcaagatcgtgaaacaggccggcgtggccacaaagggccccaacgagtatatccaagagattgagttcgagaatctgagccccggcagcgtgatcatcttcagagtgtccctggatcctcacgctcaggtggccgtgggcatcctgagaaatcacctgacacagttcagcccacacttcaagagcggaagcctggccgtggacaacgccgatcctatcctgaagatccccttcgcctctctggcctccagactgacactggctgagctgaaccagatcctgtacagatgcgagtccgaagagaaagaggatggcggaggctgctacgacatccccaattggagcgccctgaagtatgccggactgcagggactgatgtctgtgctggccgagatcagacccaagaacgacctgggacaccccttctgcaacaacctgagatccggcgactggatgatcgactacgtgtccaacagactgatcagcagatccggcacaatcgccgaagtcggcaaatggctgcaggccatgttcttctacctgaagcagatccctcggtatctgatcccctgctacttcgacgccatcctgatcggcgcctacaccacactgctggataccgcctggaagcagatgtccagcttcgtgcagaacggcagcaccttcgtgaagcacctgtctctgggaagcgtgcagctgtgtggcgtgggcaaatttcccagcctgcctatcctgtctcctgcactgatggacgtgccctaccggctgaatgagatcaccaaagaaaaagagcagtgctgcgtcagcctggctgctggcctgcctcatttttccagcggcatcttccggtgttggggcagagacacctttattgccctgagaggcatcctgctgattaccggcagatacgtggaagcccggaacatcatcctggcctttgccggcacactgcggcacggactgattcctaatctgctcggcgagggcatctacgccagatacaactgcagagatgccgtgtggtggtggctccagtgcatccaggactactgcaagatggtgcccaacggcctggacatcctgaagtgccctgtgtccagaatgtaccctaccgacgatagcgcccctctgcctgccggaacacttgaccagcctctgttcgaagtgattcaagaggccatgcagaaacacatgcagggaatccagtttcgcgagcggaatgccggacctcagatcgacagaaacatgaaggatgagggcttcaacatcaccgctggcgtggacgaagagacaggctttgtgtacggcggcaaccggttcaattgcggcacctggatggacaagatgggcgagtctgaccgggccagaaacagaggaattcccgccacacctagagatggcagcgctgtggaaatcgtgggcctgtctaagtctgctgtgcggtggctgctcgaactgagcaagaagaatatctttccgtaccacgaagtgaccgtgaagcggcacggcaaggccatcaaggtgtcctacgacgagtggaacagaaagatccaggacaacttcgaaaagctgttccatgtgtctgaggaccccagcgacctgaacgaaaagcaccccaacctggtgcacaagcgcggcatctacaaggacagctacggcgcctcttctccttggtgcgattaccagctgcggcccaacttcaccattgccatggtggttgcccctgagctgttcaccacagagaaggcctggaaggccctggaaatcgccgagaagaaactgctgggccctctgggcatgaagacactggaccccgacgacatggtgtactgcggaatctacgacaacgccctggataacgacaactacaatctggccaaggggttcaattaccatcagggacccgagtggctgtggcctatcggctatttcctgcgggccaagctgtacttctccagactgatgggccctgagacaaccgccaagacaatcgtgctcgtgaagaacgtgctgagccggcactatgtgcacctggaaagaagcccctggaagggactgcccgagctgaccaatgagaacgcccagtactgccccttcagctgcgaaacacaggcctggtctatcgccaccatcctggaaaccctgtacgacctgtgagtttaaacgcggccgcaataaaagatctttattttcattagatctgtgtgttggttttttgtgtgt

SEQ ID NO: 51 - DNA, 72 bp, synthetic construct
  misc_feature 1..72: note = HS-CRM8
  source 1..72: mol_type = other DNA
gggggaggctgctggtgaatattaaccaaggtcaccccagttatcggaggagcaaacaggggctaagtccac

SEQ ID NO: 52 - DNA, 231 bp, synthetic construct
  misc_feature 1..231: note = short EF1alpha promoter
  source 1..231: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 53 - DNA, 262 bp, synthetic construct
  misc_feature 1..262: note = HSE promoter
  source 1..262: mol_type = other DNA
cgcgccgggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggt

SEQ ID NO: 54 - protein, 5 aa, synthetic construct
  REGION 1..5: note = N-ter amino acids of d1b4 (without Met)
GSFQY

SEQ ID NO: 55 - protein, 5 aa, synthetic construct
  REGION 1..5: note = N-ter amino acids of d1b5 (without Met)
EKSGG

SEQ ID NO: 56 - protein, 5 aa, synthetic construct
  REGION 1..5: note = N-ter amino acids of d1b6 (without Met)
ILRVG

SEQ ID NO: 57 - protein, 5 aa, synthetic construct
  REGION 1..5: note = N-ter amino acids of d1b7 (without Met)
GADNH

SEQ ID NO: 58 - protein, 5 aa, synthetic construct
  REGION 1..5: note = N-ter amino acids of d1b8 (without Met)
LDCVT

SEQ ID NO: 59 - DNA, 20 bp, synthetic construct
  misc_feature 1..20: note = forward primer
  source 1..20: mol_type = other DNA
ctgaagctgtgggagttctt

SEQ ID NO: 60 - DNA, 23 bp, synthetic construct
  misc_feature 1..23: note = reverse primer
  source 1..23: mol_type = other DNA
ctcttggtcactcttctgttctc

SEQ ID NO: 61 - DNA, 21 bp, synthetic construct
  misc_feature 1..21: note = forward primer
  source 1..21: mol_type = other DNA
aaaacgagcagtgacgtgagc

SEQ ID NO: 62 - DNA, 20 bp, synthetic construct
  misc_feature 1..20: note = reverse primer
  source 1..20: mol_type = other DNA
ttcagtcatgctgctagcgc

SEQ ID NO: 63 - protein, 64 aa, synthetic construct
  REGION 1..64: note = N-terminal region of the GDE enzyme of Candida glabrata
KFYKKIIKTSFHEDDHIDLDIYVPGTYCFYLSFKNDKDELETTRKFYFVVLPILSVNDKFIPLN

SEQ ID NO: 64 - protein, 65 aa, synthetic construct
  REGION 1..65: note = N-terminal region of the human GDE enzyme
KFRSLDWENPTEREDDSDKYCKLNLQQSGSFQYYFLQGNEKSGGGYIVVDPILRVGADNHVLPLD

(19) United States
(12) Patent Application Publication
(10) Pub. No.: US 2026/0242762 A1
(43) Pub. Date: Aug. 20, 2026

(54) N-TERMINAL TRUNCATED GDE FOR THE TREATMENT OF GLYCOGEN STORAGE DISEASE III

(71) Applicants: GENETHON, EVRY-COURCOURONNES (FR); INSTITUT NATIONAL DE LA SANTÉ ET DE LA RECHERCHE MÉDICALE, PARIS (FR); UNIVERSITE D'EVRY VAL D'ESSONNE, EVRY-COURCOURONNES (FR)

(72) Inventors: ANTOINE GARDIN, PARIS (FR); GIUSEPPE RONZITTI, FONTAINEBLEAU (FR); JÉRÉMY ROUILLON, SAINT-FARGEAU-PONTHIERRY (FR)

(21) Appl. No.: 18/872,298

(22) PCT Filed: Jun. 9, 2023
(86) PCT No.: PCT/EP2023/065474

§ 371 Date: Dec. 6, 2024

(30) Foreign Application Priority Data
Jun. 9, 2022 (EP) 22305841.3

(51) Int. Cl. C12N 9/44; A61K 38/00; A61P 1/16; C12N 9/10; C12N 15/86

(52) U.S. Cl. CPC C12N 9/2451; A61P 1/16; C12N 9/1051; C12N 15/86; A61K 38/00; C12N 2750/14143; C12Y 204/01025; C12Y 302/01033

(57) ABSTRACT

The present invention relates to a functional N-terminal truncated GDE polypeptide for the treatment of glycogen storage disease III.

### BACKGROUND OF THE INVENTION

Mutations in the AGL gene cause genetic deficiency of glycogen debranching enzyme (GDE), or “amylo-alpha-1,6-glucosidase, 4-alpha-glucanotransferase”, an enzyme involved in glycogen degradation. GDE has two independent catalytic activities which occur at different sites on the protein: a 4-alpha-glucotransferase activity and an amylo-1,6-glucosidase activity. Genetic deficiency of GDE causes an incomplete glycogenolysis in glycogen storage disease III (GSDIII), resulting in accumulation of abnormal glycogen with short outer chain in various organs, mostly liver and muscle. The disease is characterized by hepatomegaly, hypoglycemia, short stature, variable myopathy and cardiomyopathy.

Most patients have GSDIII involving both liver and muscle (type IIIa), while some patients (~15 percent) have only liver involvement (type IIIb). Liver symptoms normally occur in childhood. Liver cirrhosis and hepatocellular carcinoma have been reported in some cases (Chen et al., 2009, Scriver's Online Metabolic & Molecular Bases of inherited Disease, New York: McGraw-Hill; Kishnani et al., 2010, Genet Med 12, 446-463). Muscle weakness could be present during childhood. It becomes more prevalent in adults with onset in the third or fourth decade. There is significant morbidity from progressive muscle weakness and patients in later stages can become wheel chair bound. Patients can also develop cardiomyopathy. There is significant clinical variability in the severity of the symptoms that these patients develop. The progressive myopathy and/or cardiomyopathy and/or peripheral neuropathy are major causes of morbidity in adults (Kishnani et al., 2010, Genet Med 12, 446-463; Cornelio et al., 1984, Arch Neurol 41, 1027-1032; Coleman et al., 1992, Ann Intern Med 116, 896-900). Reports of possible neurological manifestations associated with the disease derive from clinicians working with GSDIII patients, who reported attention fluctuations, deficiencies in executive functions and impaired emotional skills (Michon et al., 2015, J Inherit Metab Dis, 38 (3): 573-580). Accordingly, in the GDE^{-/-} mouse model of the disease, an extensive accumulation of glycogen throughout the nervous system was documented (Pagliarani et al., 2014, Biochim Biophys Acta, 1842 (11): 2318-2328; Liu et al., 2014, Mol Genet Metab, 111 (4): 467-476) although a careful characterization of the phenotype associated with the accumulation of glycogen is still missing. Current treatment is symptomatic, and there is no effective therapy for the disease. Hypoglycemia can be controlled by frequent meals high in carbohydrates with cornstarch supplements or nocturnal gastric drip feedings. Patients with myopathy have been treated with a diet high in protein during the daytime plus overnight enteral infusion. In some patients transient improvement in symptoms has been documented, but there are no systemic studies or long-term data demonstrating that the high protein diet prevents or treats the progressive myopathy (Kishnani et al., 2010, Genet Med 12, 446-463). These approaches do little to alter the long term course and morbidity of these diseases.

Therefore, there is still a need for a long-term treatment of GSDIII. Gene therapy aiming to stably replace the GDE protein in the affected tissues appears as a potential therapeutic approach. However, the large size of the GDE transgene constitutes a major impediment since it cannot fit the size limit of most gene therapy vectors. Indeed, the human AGL gene is 85 kb in length and composed of 35 exons, encoding a 7.4-kb mRNA that includes a 4599-bp coding region and a 2371-bp 3′ untranslated sequence to express a 175 kDa GDE protein (Bao Y et al., 1996, Genomics., 38 (2): 155-65). This constitutes a real issue since the minimum size of a GDE expression cassette (including for example at least a promoter, the GDE coding sequence, a polyA signal and the two ITRs for an AAV vector) would be larger than 5 kb, the genome size limit that can be packaged into an AAV gene therapy vector for in vivo gene delivery.

The inventors have previously proposed the use of dual AAV vectors to overcome this size limitation. Following this approach, two vectors, each containing a portion of the large transgene coding sequence, are used to transduce the same cell. Although the use of dual AAV vectors is promising, it would be preferable to provide a gene therapy strategy implementing only one viral vector for both economic and practical reasons.

In patent applications WO2020/030661 and PCT/EP2021/073309, the present inventors described another approach based on the use of a truncated GDE polypeptide that fits in a single viral vector. Yet, alternatives to the truncated GDE polypeptides previously described in WO2020/030661 and PCT/EP2021/073309 are desirable.

### SUMMARY OF THE INVENTION

The present invention relates to a functional truncated GDE polypeptide, wherein said functional truncated GDE polypeptide comprises a deletion with respect to a reference functional full-length human GDE sequence, and wherein said deletion consists of the deletion of amino acids in the N-terminal part of the reference functional full-length human GDE sequence in such a way that the first six amino acids at the N-terminus of the functional truncated GDE polypeptide are:

(SEQ ID NO: 7)
-MGSFQY;
(SEQ ID NO: 8)
-MEKSGG;
(SEQ ID NO: 9)
-MILRVG;
(SEQ ID NO: 10)
-MGADNH;
or
(SEQ ID NO: 11)
-MLDCVT.

In a particular embodiment, the deletion consists of the deletion of amino acids in the N-terminal part of the reference functional full-length human GDE sequence in such a way that the first six amino acids at the N-terminus of the functional truncated GDE polypeptide are MEKSGG (SEQ ID NO:8) or MILRVG (SEQ ID NO:9), preferably wherein the first six amino acids at the N-terminus of the functional truncated GDE polypeptide are MEKSGG (SEQ ID NO:8).

In a particular embodiment, the reference functional full-length human GDE has an amino acid sequence as shown in SEQ ID NO:1, SEQ ID NO:2, SEQ ID NO:3, SEQ ID NO:4, SEQ ID NO:5 or SEQ ID NO: 6, or has an amino acid sequence having at least 80, 85, 90, 95, 96, 97, 98 or at least 99 percent sequence identity to SEQ ID NO:1, SEQ ID NO:2, SEQ ID NO:3, SEQ ID NO:4, SEQ ID NO:5 or SEQ ID NO: 6. In a particular embodiment, the reference functional full-length human GDE has an amino acid sequence as shown in SEQ ID NO:1 or SEQ ID NO:4, preferably SEQ ID NO:1, or has an amino acid sequence having at least 80, 85, 90, 95, 96, 97, 98 or at least 99 percent sequence identity to SEQ ID NO: 1 or SEQ ID NO:4, preferably to SEQ ID NO:1.

In a particular embodiment, the functional truncated GDE polypeptide further comprises a deletion or a combination of deletions with respect to the reference functional full-length human GDE sequence, such as a deletion or a combination of deletions in the C-terminal part of the GDE sequence, or a deletion or a combination of deletions in the central domain of the GDE sequence. In a particular embodiment, the functional truncated GDE polypeptide further comprises a deletion or a combination of deletions with respect to SEQ ID NO:1, SEQ ID NO:2, SEQ ID NO:3, SEQ ID NO:4, SEQ ID NO:5 or SEQ ID NO:6, wherein the deletion(s) is (are) selected from any deletion referred to as 41, 42, 43, 44, 45, 46, and 47 in table 2 below.

In a particular embodiment, the functional truncated GDE polypeptide has an amino acid sequence as shown in SEQ ID NO:12-16 or has an amino acid sequence having at least 80, 85, 90, 95, 96, 97, 98 or at least 99 percent sequence identity to SEQ ID NO:12-16. In a particular embodiment, the functional truncated GDE polypeptide has an amino acid sequence as shown in SEQ ID NO:13 or 14, preferably SEQ ID NO:13, or has an amino acid sequence having at least 80, 85, 90, 95, 96, 97, 98 or at least 99 percent sequence identity to SEQ ID NO:13 or 14, preferably SEQ ID NO: 13.

The present invention also relates to a nucleic acid molecule encoding the functional truncated GDE polypeptide of the invention.

The present invention also relates to an expression cassette, comprising, preferably in this order:
a promoter;
optionally, an intron;
the nucleic acid molecule of claim of the invention; and
a polyadenylation signal.

Another aspect of the invention relates to a vector, in particular a viral vector, comprising the nucleic acid molecule or the expression cassette of the invention. In a particular embodiment, the vector is an AAV vector.

The present invention also relates to an isolated cell transformed with the nucleic acid molecule, the expression cassette or the vector of the invention, wherein the cell is in particular a liver cell, a muscle cell, a cardiac cell or CNS cell.

The invention also relates to the functional truncated GDE polypeptide, the nucleic acid molecule, the expression cassette, the vector, or the cell as defined above, for use as a medicament. The invention also relates to the functional truncated GDE polypeptide, the nucleic acid molecule, the expression cassette, the vector, or the cell as defined above, for use in a method for treating a disease caused by a mutation in the AGL gene encoding GDE. In a further particular embodiment, the invention relates to the functional truncated GDE polypeptide, the nucleic acid molecule, the expression cassette, the vector, or the cell as defined above, for use in a method for treating GSDIII (Cori disease).

### LEGENDS TO THE FIGURES

FIG. 1. (A) N-terminal region of the GDE enzyme in Candida glabrata (above) and in humans (below). Truncation sites of the “Δ1b2” and “Δ1b3” proteins, as well as truncation sites of the new truncated proteins (“Δ1b4” to “Δ1b8”) are shown. (B) Experimental design of intramuscular injection of rAAV vectors containing the truncated forms of GDE into GSDIII mice. The truncated “Δ1b3” protein and the full-length GDE (FS) were used as controls.

FIG. 2. Glycogen quantification in left tibialis anterior muscle (TA) 1 month after intramuscular injection of rAAV vector containing a truncated GDE polypeptide (Δ1b5). The truncated Δ1b3 polypeptide and the full-length GDE were used as positive controls. KO and WT mice were injected with saline (PBS) as negative controls.

FIG. 3: Western blot analysis of GDE expression from left anterior tibial muscle (TA) lysate 1 month after intramuscular injection of rAAV vector containing a truncated GDE polypeptide (Δ1b5). The truncated Δ1b3 polypeptide and the full-length GDE were used as positive controls. KO and WT mice were injected with saline (PBS) as negative controls.

FIG. 4: Quantification of GDE protein expression, based on the Western Blot of FIG. 3.

FIG. 5: Quantification of Vector Genome Copy Number (VGCN) by quantitative PCR in the tibial anterior (TA) muscle of mice injected with saline (PBS) or an rAAV vector expressing either a truncated GDE polypeptide (Δ1b3 or Δ1b5) or the full length GDE protein (GDE full size).

FIG. 6: Partial correction of muscle and heart impairment in the Agl^{-/-} mouse model after injection of arAAV-Δ1b3-GDE vector. (A) 4-month-old male Agl^{-/-} mice were injected in the tail vein with arAAV vector encoding Δ1b3-GDE, at the dose of 1×10¹⁴ vg/kg. PBS-injected Agl^{+/+} and Agl^{-/-} mice were used as controls. (B) Glycogen content measured in heart and triceps at euthanasia. (C) Wire-hang test expressed as number of falls per minute, performed 3 months after vector injection. Statistical analyses were performed by one-way ANOVA (*p<0.05, ** p<0.01 or *** p<0.001 vs. PBS-injected Agl^{-/-} mice; #p<0.05, ** p<0.01 or *** p<0.001 vs. PBS-injected Agl^{+/+} mice; n=4 mice per group). All data are shown as mean±SEM.

FIG. 7: Complete correction of muscle and heart impairment in the Agl^{-/-} mouse model after injection of a rAAV-Δ1b5-GDE vector. (A) 4-month-old male Agl^{-/-} mice were injected in the tail vein with a rAAV vector encoding Δ1b5-GDE, at the dose of 1×10¹⁴ vg/kg. PBS-injected Agl^{+/+} and Agl^{-/-} mice were used as controls. (B) Glycogen content measured in heart and triceps at euthanasia. (C) Wire-hang test expressed as number of falls per minute, performed 3 months after vector injection. Statistical analyses were performed by one-way ANOVA (*p<0.05, ** p<0.01 or *** p<0.001 vs. PBS-injected Agl^{-/-} mice; * p<0.05, *** p<0.01 or *** p<0.001 vs. PBS-injected Agl^{+/+} mice; n=7-9 mice per group coming from two independent experiments). All data are shown as mean±SEM.

FIG. 8: Correction of muscle and heart impairment in the Agl^{-/-} rat model after injection of a rAAV-Δ1b5-GDE vector. (A) 6-week-old male Agl^{-/-} rats were injected in the tail vein with a rAAV vector encoding the Δ1b5-GDE, at the dose of 1×10¹⁴ vg/kg. PBS-injected Agl^{-/-} and Agl^{+/+} rats were used as controls. (B, D) Glycogen content measured in heart (B) and triceps (D) at euthanasia. (C, E) Histological analysis of heart (C) and triceps (E) with HPS and PAS staining. Representative images are shown. Statistical analyses were performed by one-way ANOVA (*p<0.05, ** p<0.01 or *** p<0.001 vs. PBS-injected Agl^{-/-} rats; * p<0.05, ##p<0.01 or ###p<0.001 vs. PBS-injected Agl^{+/+} rats; n=5 rats per group). All data are shown as mean±SEM.

FIG. 9: Clearance of glycogen in human induced pluripotent stem cells (hiPSC)—derived skeletal muscle cells treated with a rAAV-Δlb5-GDE vector. (A) Transduction protocol of AGL knocked-out (GSDIII^{CRISPR}) hiPSC-derived skeletal myoblasts and isogenic controls with an rAAV vector expressing either GFP or Δ1b5-GDE at a MOI of either 15 000 or 75 000. (B) Expression of specific myogenic markers (desmin, myosin heavy chain (MHC) and titin) and GFP by immunofluorescence analysis, after rAAV transduction at MOI 75 000 in GSDIII^{CRISPR} hiPSC-derived skeletal myotubes. Representative images are shown. (C) Glycogen content normalized to the number of viable cells measured in GSDIII^{CRISPR} hiPSC-derived skeletal myotubes and isogenic controls (CTRL1) transduced as described above. (D) PAS staining on GSDIII^{CRISPR} hiPSC-derived skeletal myotubes and isogenic controls (CTRL1) transduced as described above. Representative images are shown. (E) Quantification of PAS staining. Statistical analyses were performed by one-way ANOVA (*p<0.05, ** p<0.01 or *** p<0.001 vs. GSDIII^{CRISPR} cells transduced with rAAV-GFP; * p<0.05, ##p<0.01 or *** p<0.001 vs. CTRL1 cells, three replicates for experiments in panel B-C and 5 replicates for experiments in panel D-E). All data are shown as mean±SEM.

FIG. 10: Improved productivity and quality of vectors encoding truncated Δ1b5-GDE. (A) Schematic representation of the two expression cassettes evaluated, with their respective size. (B) Small scale rAAV productions (50 mL culture) were performed in triplicate for each vector and viral titers were measured both before (bulk) and after purification (final product) for each triplicate. (C) rAAV vector DNA was extracted and loaded on a 1% Agarose gel to assess genome integrity. Expected genome size range from 5.0 to 5.3 kb. (D) Analytical ultracentrifugation analysis of the proportion of full (right peak in the curve) and empty (left peak in the curve) particles. Statistical analyses were performed by one-way ANOVA (*p<0.05, ** p<0.01 or *** p<0.001 vs. miniCMV GDE full length, experiment performed in triplicate). All data are shown as mean±SEM.

### DETAILED DESCRIPTION OF THE INVENTION

In patent applications WO2020/030661 and PCT/EP2021/073309, the present inventors have demonstrated that it is possible to use a truncated GDE polypeptide having a size compatible for encapsidation in an AAV vector while retaining its enzymatic activity.

Despite the lack of knowledge regarding the three-dimensional structure of the GDE protein, the present inventors have identified new N-terminal truncated GDE polypeptides having a high protein expression level and a very good efficacy in terms of glycogen reduction in GSDIII mice.

The present invention thus relates to a functional N-terminal truncated GDE polypeptide. This polypeptide can advantageously be used in a method for treating a disease caused by a mutation in the AGL gene encoding GDE, in particular in a method for treating GSDIII (Cori disease).

### 1-N-Terminal Truncated GDE Polypeptide

The truncated GDE polypeptide according to the present invention is a functional GDE polypeptide whose coding sequence is small enough to be efficiently packaged into a gene therapy vector, in particular into a single AAV vector.

By “functional” GDE polypeptide is meant a polypeptide that retains, at least in part, at least one of the enzymatic activities of the GDE protein, preferably all of the enzymatic activities of the GDE protein. As a consequence, the functional GDE polypeptide implemented in the present invention is able to rescue glycogen accumulation and muscle strength in vivo. As defined herein, GDE enzymatic activities are a 4-alpha-glucotransferase activity and an amylo-1,6-glucosidase activity, involved in glycogen degradation. The transferase activity of GDE relocates three glucose units of glycogen from one chain to another. This leaves one glucose unit at the branch point, which is subsequently released as glucose by the glucosidase activity. In a particular embodiment, the functional GDE polypeptide of the invention has the same functionality as a full-length GDE polypeptide, in particular as a full-length human GDE polypeptide. For example, a functional GDE polypeptide of the invention may have an activity of at least 5%, 10%, 20%, 30%, 40%, 50%, 60%, 70%, 80%, 90%, 95%, or at least 99% in relation to one, preferably both, enzymatic activities described above, or at least 100%, as compared to a full-length human GDE protein, in particular the full-length human GDE protein of SEQ ID NO:1, SEQ ID NO: 2, SEQ ID NO:3, SEQ ID NO:4, SEQ ID NO:5, or SEQ ID NO:6. The activity of the GDE protein of the invention may even be of more than 100%, such as of more than 110%, 120%, 130%, 140%, 150%, 200%, 500%, 700%, or even more than 1000% of the activity of a full-length human GDE protein, in particular the full-length human GDE protein of SEQ ID NO:1, SEQ ID NO:2, SEQ ID NO:3, SEQ ID NO:4, SEQ ID NO:5, or SEQ ID NO:6. In a particular embodiment, the functional GDE polypeptide of the invention has the same functionality as a full-length GDE polypeptide, in particular as a full-length human GDE polypeptide in muscular tissues such as heart or quadriceps. For example, a functional GDE polypeptide of the invention may have an activity in muscular tissues such as heart or quadriceps of at least 50%, 60%, 70%, 80%, 90%, 95%, or at least 99% in relation to one, preferably both, enzymatic activities described above, or at least 100%, as compared to a full-length human GDE protein, in particular the full-length human GDE protein of SEQ ID NO:1, SEQ ID NO:2, SEQ ID NO:3, SEQ ID NO:4, SEQ ID NO:5, or SEQ ID NO:6. The activity in muscular tissues such as heart or quadriceps of the GDE protein of the invention may even be of more than 100%, such as of more than 110%, 120%, 130%, 140%, 150%, 200%, 500%, 700%, or even more than 1000% of the activity of a full-length human GDE protein, in particular the full-length human GDE protein of SEQ ID NO:1, SEQ ID NO:2, SEQ ID NO:3, SEQ ID NO:4, SEQ ID NO:5, or SEQ ID NO:6.

By “functional” GDE polypeptide is meant a GDE polypeptide that is not pathological. In particular, the functional GDE polypeptide of the invention is not a GDE polypeptide found in a patient with GSDIII (Cori disease), such as GSDIIIa or GSDIIIb.

A skilled person is readily able to determine whether a polypeptide is a functional GDE polypeptide. Suitable methods would be apparent to those skilled in the art. For example, one suitable in vitro method involves inserting a nucleic acid encoding a polypeptide into a vector, such as a plasmid or viral vector, transfecting or transducing host cells, such as 293T or HeLa cells, or other cells such as Huh7, with the vector, and assaying for GDE activity. Suitable methods are described in more details in the experimental part below. For example, GDE activity may be determined by measuring the glucose produced after incubating homogenized tissues or cell extracts, previously transfected with a vector expressing a functional GDE polypeptide with limit dextrin (glycogen phosphorylase-digested glycogen). Other methods include testing the GDE efficacy by evaluating muscle strength of treated GDE-KO animals by wire-hang after administration of the vectors, such as after one, two or three months after administration, by evaluating the rescue of glycogen accumulation in muscle and/or cardiac tissue and/or by evaluating the normalization of glycemia in treated GDE-KO animals after administration of the vectors, such as after one, two or three months after administration. Furthermore, GDE expression can be evaluated in tissues of a GDE KO animal after administration of the vectors, such as after one, two or three months after administration, by western blot.

In the context of the present invention, “a reference full-length human GDE sequence” encompasses all native isoforms of human GDE. Bao and colleagues (Genomics, 1997, 38, 155-165) identified the presence of six transcript variants encoding for three GDE protein isoforms. Transcript variants 1~4 encode for the same protein, namely GDE isoform 1. Transcript variants 5 and 6 encode for GDE isoforms 5 and 6 respectively.

In the context of the present invention, “a reference full-length human GDE sequence” does not encode a pathological GDE polypeptide. In particular, the reference full-length human GDE sequence does not encode a pathological variant found in a patient with GSDIII, comprising mutation(s), deletion(s) or insertion(s) as compared to the wild-type non-pathological full-length human GDE sequence.

The term “reference full-length human GDE polypeptide” thus encompasses all native isoforms of human GDE including the precursor form, as well as modified or mutated by insertion(s), deletion(s) and/or substitution(s) GDE proteins or fragments thereof that are functional derivatives of GDE. In particular, the reference full-length human GDE sequence is selected from the group consisting of SEQ ID NO: 1 (corresponding to wild-type GDE isoform 1, UniProtKB identifier: P35573-1), SEQ ID NO:4 (corresponding to a variant of GDE isoform 1), SEQ ID NO:2 (corresponding to wild-type GDE isoform 5, UniProtKB identifier: P35573-2), SEQ ID NO:5 (corresponding to a variant of GDE isoform 5), SEQ ID NO: 3 (corresponding to wild-type GDE isoform 6, UniProtKB identifier: P35573-3), and SEQ ID NO: 6 (corresponding to a variant of GDE isoform 6).

In a particular embodiment, the reference full-length human GDE has at least 80, 85, 90, 95, 96, 97, 98 or at least 99 percent sequence identity to SEQ ID NO:1, SEQ ID NO:2, SEQ ID NO:3, SEQ ID NO:4, SEQ ID NO:5, or SEQ ID NO:6, in particular to SEQ ID NO:1, SEQ ID NO:2 or SEQ ID NO:3. In a particular embodiment, the reference full length human GDE has at least 80, 85, 90, 95, 96, 97, 98 or at least 99 percent sequence identity to SEQ ID NO:1 or SEQ ID NO:4 and has the same length in terms of number of amino acids as SEQ ID NO:1 or SEQ ID NO:4. In a particular embodiment, the reference full length human GDE has at least 80, 85, 90, 95, 96, 97, 98 or at least 99 percent sequence identity to SEQ ID NO:2 or SEQ ID NO:5 and has the same length in terms of number of amino acids as SEQ ID NO: 2 or SEQ ID NO:5. In a particular embodiment, the reference full length human GDE has at least 80, 85, 90, 95, 96, 97, 98 or at least 99 percent sequence identity to SEQ ID NO:3 or SEQ ID NO:6 and has the same length in terms of number of amino acids as SEQ ID NO:3 or SEQ ID NO:6.

The term “identical” and declinations thereof refers to the sequence identity between two nucleic acid molecules or between two polypeptide molecules. When a position in both of the two compared sequences is occupied by the same base or the same amino acid, then the molecules are identical at that position. The percent of identity between two sequences is a function of the number of matching positions shared by the two sequences divided by the number of positions compared X 100. For example, if 6 of 10 of the positions in two sequences are matched then the two sequences are 60% identical. Generally, a comparison is made when two sequences are aligned to give maximum identity. Various bioinformatics tools known to the one skilled in the art might be used to align nucleic acid sequences such as BLAST or FASTA.

In a particular embodiment, the reference full-length human GDE sequence has an amino acid sequence as shown in SEQ ID NO:1 or SEQ ID NO:4, in particular SEQ ID NO:1 which corresponds to GDE isoform 1.

In a particular embodiment, the truncated GDE polypeptide of the invention, that is truncated with respect to a reference full-length human GDE sequence may comprise one or more additional amino acid modifications with respect to said reference full-length human GDE sequence. In particular, in addition to the deletion(s) that are further described below, the functional truncated GDE polypeptide may comprise one or more amino acid modifications such as amino acid insertion, deletion and/or substitution as compared to the reference full-length human GDE sequence. For example, the functional truncated GDE polypeptide may comprise from 1 to 10 (e.g. 1, 2, 3, 4, 5, 6, 7, 8, 9 or 10) additional amino acid modifications, in particular from 1 to 5 (e.g. 1, 2, 3, 4 or 5) additional amino acid modifications, as long as the functionality of the truncated GDE polypeptide is preserved.

The functional truncated GDE polypeptide of the invention, is a N-terminal truncated GDE polypeptide. By “N-terminal truncated GDE polypeptide” is meant a GDE polypeptide comprising a deletion with respect to a reference functional full-length human GDE sequence, wherein said deletion consists of the deletion of amino acids in the N-terminal part of the reference functional full-length human GDE sequence. The “N-terminal part” of the reference functional full-length GDE sequence refers to a region consisting of the first 280 amino acid residues (i.e. the most N-terminal 280 amino acid residues of the reference full-length GDE sequence), the first 200 amino acid residues (i.e. the most N-terminal 200 amino acid residues of the reference full-length GDE sequence), the first 150 amino acid residues (i.e. the most N-terminal 150 amino acid residues of the reference full-length GDE sequence), preferably the first 125 amino acid residues (i.e. the most N-terminal 125 amino acid residues of the reference full-length GDE sequence), most preferably the first 123 amino acid residues (i.e. the most N-terminal 123 amino acid residues of the reference full-length GDE sequence).

As detailed below, the N-terminal truncated GDE polypeptide of the invention retains a methionine as first residue at the N-terminal end.

The functional truncated GDE polypeptide of the invention comprises a deletion with respect to the reference functional full-length human GDE sequence, wherein said deletion consists of the deletion of amino acids in the N-terminal part of the reference functional full-length human GDE sequence in such a way that the first six amino acids at the N-terminus of the functional truncated GDE polypeptide are:

(SEQ ID NO: 7)
-MGSFQY;
(SEQ ID NO: 8)
-MEKSGG;
(SEQ ID NO: 9)
-MILRVG;
(SEQ ID NO: 10)
-MGADNH;
or
(SEQ ID NO: 11)
-MLDCVT.

Preferably, the functional truncated GDE polypeptide of the invention comprises a deletion with respect to the reference functional full-length human GDE sequence, wherein said deletion consists of the deletion of amino acids in the N-terminal part of the reference functional full-length human GDE sequence in such a way that the first six amino acids at the N-terminus of the functional truncated GDE polypeptide are MEKSGG (SEQ ID NO: 8).

In other words, the deletion of amino acids in the N-terminal part of the reference functional full-length human GDE leads to a truncated GDE polypeptide wherein the first six amino acids at the N-terminal end differ from the six amino acids at the N-terminal end of the reference functional full-length human GDE sequence, and wherein the first six amino acids at the N-terminal end of the truncated polypeptide are SEQ ID NO: 7, SEQ ID NO: 8, SEQ ID NO: 9, SEQ ID NO: 10 or SEQ ID NO: 11, preferably SEQ ID NO: 8.

In a particular embodiment, the functional truncated GDE polypeptide of the invention comprises a deletion with respect to the reference functional full-length human GDE sequence,
wherein the reference functional full-length human GDE sequence has an amino acid sequence selected from the group consisting in: SEQ ID NO: 1, SEQ ID NO: 2, SEQ ID NO:3, SEQ ID NO:4, SEQ ID NO: 5, or SEQ ID NO: 6;
and wherein said deletion consists of the deletion of amino acids in the N-terminal part of the reference functional full-length human GDE sequence in such a way that the first six amino acids at the N-terminus of the functional truncated GDE polypeptide are:

(SEQ ID NO: 7)
-MGSFQY;
(SEQ ID NO: 8)
-MEKSGG;
(SEQ ID NO: 9)
-MILRVG;
(SEQ ID NO: 10)
-MGADNH;
or
(SEQ ID NO: 11)
-MLDCVT.

In a preferred embodiment, the functional truncated GDE polypeptide of the invention comprises a deletion with respect to the reference functional full-length human GDE sequence,
wherein the reference functional full-length human GDE sequence has an amino acid sequence selected from the group consisting in: SEQ ID NO: 1, SEQ ID NO: 2, SEQ ID NO:3, SEQ ID NO:4, SEQ ID NO: 5, or SEQ ID NO: 6;
and wherein said deletion consists of the deletion of amino acids in the N-terminal part of the reference functional full-length human GDE sequence in such a way that the first six amino acids at the N-terminus of the functional truncated GDE polypeptide are MEKSGG (SEQ ID NO: 8).

In a particular embodiment, the functional truncated GDE polypeptide of the invention comprises a deletion with respect to the reference functional full-length human GDE sequence,
wherein the reference functional full-length human GDE sequence has an amino acid sequence selected from the group consisting in: SEQ ID NO: 1, SEQ ID NO: 2, SEQ ID NO:3, SEQ ID NO:4, SEQ ID NO: 5, or SEQ ID NO: 6;
and wherein said deletion consists of:
(i) the deletion of any amino acid between the first methionine and the sequence “GSFQY” (SEQ ID NO: 54);

(ii) the deletion of any amino acid between the first methionine and the sequence “EKSGG” (SEQ ID NO: 55);
(iii) the deletion of any amino acid between the first methionine and the sequence “ILRVG” (SEQ ID NO: 56);

(iv) the deletion of any amino acid between the first methionine and the sequence “GADNH” (SEQ ID NO: 57); or
(v) the deletion of any amino acid between the first methionine and the sequence “LDCVT” (SEQ ID NO: 58).

According to this embodiment, a deletion which consists of the deletion of any amino acid between the first methionine and the sequence “GSFQY” means that all consecutive amino acids between the first methionine at the N-terminal end and the sequence “GSFQY” are deleted, while said first methionine and said sequence “GSFQY” (SEQ ID NO:54) are not deleted.

According to this embodiment, a deletion which consists of the deletion of any amino acid between the first methionine and the sequence “EKSGG” means that all consecutive amino acids between the first methionine at the N-terminal end and the sequence “EKSGG” are deleted, while said first methionine and said sequence “EKSGG” (SEQ ID NO:55) are not deleted.

According to this embodiment, a deletion which consists of the deletion of any amino acid between the first methionine and the sequence “ILRVG” means that all consecutive amino acids between the first methionine at the N-terminal end and the sequence “ILRVG” are deleted, while said first methionine and said sequence “ILRVG” (SEQ ID NO:56) are not deleted.

According to this embodiment, a deletion which consists of the deletion of any amino acid between the first methionine and the sequence “GADNH” means that all consecutive amino acids between the first methionine at the N-terminal end and the sequence “GADNH” are deleted, while said first methionine and said sequence “GADNH” (SEQ ID NO:57) are not deleted.

According to this embodiment, a deletion which consists of the deletion of any amino acid between the first methionine and the sequence “LDCVT” means that all consecutive amino acids between the first methionine at the N-terminal end and the sequence “LDCVT” are deleted, while said first methionine and said sequence “LDCVT” (SEQ ID NO:58) are not deleted.

In a preferred embodiment, the functional truncated GDE polypeptide of the invention comprises a deletion with respect to the reference functional full-length human GDE sequence, -wherein the reference functional full-length human GDE sequence has an amino acid sequence selected from the group consisting in: SEQ ID NO: 1, SEQ ID NO: 2, SEQ ID NO:3, SEQ ID NO:4, SEQ ID NO: 5, or SEQ ID NO: 6;
and wherein said deletion consists of the deletion of any amino acid between the first methionine and the sequence “EKSGG”.

In a particular embodiment, the functional truncated GDE polypeptide of the invention comprises a deletion with respect to the reference functional full-length human GDE sequence,
wherein the reference functional full-length human GDE sequence has an amino acid sequence of SEQ ID NO: 1 or SEQ ID NO:4, or a functional variant of SEQ ID NO:1 or SEQ ID NO:4 having the same length,
wherein said deletion consists of:
(i) the deletion of amino acids at positions 2-88, and wherein amino acids at position 1 and at position 89 are not deleted;
(ii) the deletion of amino acids at positions 2-99, and wherein amino acids at position 1 and at position 100 are not deleted;
(iii) the deletion of amino acids at positions 2-111, and wherein amino acids at position 1 and at position 112 are not deleted;
(iv) the deletion of amino acids at positions 2-115, and wherein amino acids at position 1 and at position 116 are not deleted; or
(v) the deletion of amino acids at positions 2-123, and wherein amino acids at position 1 and at position 124 are not deleted.

In a preferred embodiment, the functional truncated GDE polypeptide of the invention comprises a deletion with respect to the reference functional full-length human GDE sequence,
wherein the reference functional full-length human GDE sequence has an amino acid sequence of SEQ ID NO: 1 or SEQ ID NO:4, or a functional variant of SEQ ID NO:1 or SEQ ID NO:4 having the same length,
wherein said deletion consists of the deletion of amino acids at positions 2-99, and wherein amino acids at position 1 and at position 100 are not deleted.

In a particular embodiment, the functional truncated GDE polypeptide of the invention comprises one and only one deletion in the N-terminal part of the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein the N-terminal part corresponds to a region consisting of the first 280 amino acid residues of the reference functional full length human GDE sequence,
and wherein said deletion in the N-terminal part of the reference functional full-length human GDE sequence consists of:
(i) the deletion of amino acids at positions 2-88 of the reference functional full-length human GDE sequence;
(ii) the deletion of amino acids at positions 2-99 of the reference functional full-length human GDE sequence;
(iii) the deletion of amino acids at positions 2-111 of the reference functional full-length human GDE sequence;
(iv) the deletion of amino acids at positions 2-115 of the reference functional full-length human GDE sequence;
(v) the deletion of amino acids at positions 2-123 of the reference functional full-length human GDE sequence.

In a preferred embodiment, the functional truncated GDE polypeptide of the invention comprises one and only one deletion in the N-terminal part of the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein the N-terminal part corresponds to a region consisting of the first 280 amino acid residues of the reference functional full length human GDE sequence,
and wherein said deletion in the N-terminal part of the reference functional full-length human GDE sequence consists of the deletion of amino acids at positions 2-99 of the reference functional full-length human GDE sequence.

By “one and only one deletion” is meant that there is one deletion in the N-terminal part which corresponds to a region consisting of the first 280 amino acid residues of the reference functional full length human GDE sequence (i.e. the most N-terminal 280 amino acid residues of the reference full-length GDE sequence). For the sake of clarity, a functional truncated GDE which comprises one and only one deletion of amino acids at positions 2-88 of the N-terminal part of the reference functional full-length human GDE sequence, corresponds to truncated polypeptide:
wherein amino acids at positions 2-88 of the reference functional full-length sequence are deleted, and wherein amino acids at positions 1 and 89-280 are not deleted.

In a particular embodiment, the functional truncated GDE polypeptide of the invention comprises one and only one deletion with respect to the reference functional full-length human GDE sequence,
wherein the reference functional full-length human GDE sequence is selected from the group consisting of SEQ ID NO:1 to SEQ ID NO:6,
and wherein the deletion is referred to as Δ1b4, Δ1b5, Δ1b6, Δ1b7, Δ1b8 in table 1, preferably Δ1b5:

TABLE 1

| | Position of the | Position of the | Position of the |
|---|---|---|---|
| | deleted amino | deleted amino | deleted amino |
| | acids with respect | acids with respect | acids with respect |
| | to SEQ ID NO: 1 | to SEQ ID NO: 2 | to SEQ ID NO: 3 |
| Deletion | or SEQ ID NO: 4 | or SEQ ID NO: 5 | or SEQ ID NO: 6 |
| Δ1b4 | 2-88 | 2-71 | 2-72 |
| Δ1b5 | 2-99 | 2-82 | 2-83 |
| Δ1b6 | 2-111 | 2-94 | 2-95 |
| Δ1b7 | 2-115 | 2-98 | 2-99 |
| Δ1b8 | 2-123 | 2-106 | 2-107 |

For the sake of clarity, Table 1 should be understood as follows. When the reference full-length GDE sequence is SEQ ID NO:1 or SEQ ID NO:4, the functional “Δlb4” truncated GDE polypeptide corresponds to a functional truncated GDE polypeptide derived from SEQ ID NO:1 or SEQ ID NO:4, which is deleted of all consecutive amino acids from position 2-88 with respect to SEQ ID NO: 1 or SEQ ID NO: 4.

### “ΔLb4” Truncated Polypeptide

In a particular embodiment, the functional truncated GDE polypeptide of the invention comprises a deletion with respect to the reference functional full-length human GDE sequence, wherein said deletion consists of the deletion of amino acids in the N-terminal part of the reference functional full-length human GDE sequence in such a way that the first six amino acids at the N-terminus of the functional truncated GDE polypeptide are MGSFQY (SEQ ID NO: 7).

In a particular embodiment, the functional truncated GDE polypeptide of the invention comprises a deletion with respect to the reference functional full-length human GDE sequence,
wherein the reference functional full-length human GDE has an amino acid sequence as shown in SEQ ID NO:1 or SEQ ID NO:4, or has an amino acid sequence having at least 80, 85, 90, 95, 96, 97, 98 or at least 99 percent sequence identity to SEQ ID NO:1 or SEQ ID NO:4; and
wherein said deletion consists of the deletion of amino acids in the N-terminal part of the reference functional full-length human GDE sequence in such a way that the first six amino acids at the N-terminus of the functional truncated GDE polypeptide are “MGSFQY” (SEQ ID NO: 7).

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-88 of the reference functional full-length GDE sequence, and
wherein the amino acid at position 1 and the amino acid at position 89 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-88 of the reference functional full-length human GDE sequence,
wherein the amino acid at position 1 of the reference functional full-length GDE sequence is not deleted, and
wherein the amino acids from positions 89 to 110, 89 to 130, 89 to 150, 89 to 170, 89 to 190, 89 to 210, 89 to 230, 89 to 250, 89 to 270, or the amino acids from positions 89 to 280 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-88 of the reference functional full-length human GDE sequence,
wherein the amino acid at position 1 of the reference functional full-length GDE sequence is not deleted, and
wherein the amino acids from positions 89 to 280 of the reference functional full-length GDE sequence are not deleted.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-88 of the reference functional full-length human GDE sequence,
and wherein said functional truncated GDE polypeptide comprises the sequence “MGSFQY” (SEQ ID NO: 7).

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-88 of the reference functional full-length human GDE sequence,
wherein said functional truncated GDE polypeptide comprises the sequence “MGSFQY” (SEQ ID NO: 7),
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-88 of the reference functional full-length human GDE sequence,
and wherein said functional truncated GDE polypeptide comprises the sequence of SEQ ID NO:17.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-88 of the reference functional full-length human GDE sequence,
wherein said functional truncated GDE polypeptide comprises the sequence of SEQ ID NO:17,
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide comprises or consists of SEQ ID NO: 12, or a functional variant thereof having at least 70% sequence identity with SEQ ID NO:12, such as at least 75% or at least 80% sequence identity, such as at least 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:12.

In a particular embodiment, the functional variant having at least 70% sequence identity with SEQ ID NO: 12, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:12, has substantially the same functionality or enzymatic activity as the GDE polypeptide of SEQ ID NO:12, in particular the same enzymatic activity involved in glycogen degradation in muscular tissues such as heart or quadriceps. In a particular embodiment, the functional variant of SEQ ID NO: 12 has substantially the same ability to rescue glycogen accumulation and muscle strength in vivo, as the GDE polypeptide of SEQ ID NO:12. In a particular embodiment, the functional variant of SEQ ID NO:12 has substantially the same level of expression as the GDE polypeptide of SEQ ID NO: 12.

In a particular embodiment, the functional variant having at least 70% sequence identity with SEQ ID NO: 12, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:12, has the same N-terminal part as SEQ ID NO:12. In a particular embodiment, the most-N terminal amino acids of the functional variant of SEQ ID NO:12 are “MGSFQY” (SEQ ID NO:7). In another particular embodiment, the most-N terminal amino acids of the functional variant of SEQ ID NO:12 correspond to the sequence of SEQ ID NO: 17

In a particular embodiment, the most N-terminal amino acids of the functional variant of SEQ ID NO:12 may consist of the amino acids at position 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:12. In other words, according to this embodiment, the functional variant of SEQ ID NO:12 does not comprise any mutation, insertion, or deletion within the region corresponding to amino acids 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:12. In a particular embodiment, the N-terminal end of the functional variant of SEQ ID NO:12 consists of the amino acids 1-50, 1-100, or 1-150 of SEQ ID NO:12. In other words, according to this embodiment, the functional variant of SEQ ID NO:12 does not comprise any mutation, insertion, or deletion within the region corresponding to amino acids 1-50, 1-100 or 1-150.

In a preferred embodiment, the functional truncated GDE polypeptide comprises or consists of SEQ ID NO: 12.

### “ΔLb5” Truncated Polypeptide

In a particular embodiment, the functional truncated GDE polypeptide of the invention comprises a deletion with respect to the reference functional full-length human GDE sequence, wherein said deletion consists of the deletion of amino acids in the N-terminal part of the reference functional full-length human GDE sequence in such a way that the first six amino acids at the N-terminus of the functional truncated GDE polypeptide are “MEKSGG” (SEQ ID NO: 8).

In a particular embodiment, the functional truncated GDE polypeptide of the invention comprises a deletion with respect to the reference functional full-length human GDE sequence,
wherein the reference functional full-length human GDE has an amino acid sequence as shown in SEQ ID NO:1 or SEQ ID NO:4, or has an amino acid sequence having at least 80, 85, 90, 95, 96, 97, 98 or at least 99 percent sequence identity to SEQ ID NO:1 or SEQ ID NO:4; and
wherein said deletion consists of the deletion of amino acids in the N-terminal part of the reference functional full-length human GDE sequence in such a way that the first six amino acids at the N-terminus of the functional truncated GDE polypeptide are “MEKSGG” (SEQ ID NO: 8).

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-99 of the reference functional full-length GDE sequence, and
wherein the amino acid at position 1 and the amino acid at position 100 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-99 of the reference functional full-length human GDE sequence,
wherein the amino acid at position 1 of the reference functional full-length GDE sequence is not deleted, and
wherein the amino acids from positions 100-110, 100 to 130, 100 to 150, 100 to 170, 100 to 190, 100 to 210, 100 to 230, 100 to 250, 100 to 270, or the amino acids from positions 100 to 280 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-99 of the reference functional full-length human GDE sequence,
wherein the amino acid at position 1 of the reference functional full-length GDE sequence is not deleted, and
wherein the amino acids from positions 100 to 280 of the reference functional full-length GDE sequence are not deleted.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-99 of the reference functional full-length human GDE sequence,
and wherein said functional truncated GDE polypeptide comprises the sequence “MEKSGG” (SEQ ID NO: 8).

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-99 of the reference functional full-length human GDE sequence,
wherein said functional truncated GDE polypeptide comprises the sequence “MEKSGG” (SEQ ID NO: 8),
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-99 of the reference functional full-length human GDE sequence,
and wherein said functional truncated GDE polypeptide comprises the sequence of SEQ ID NO:18.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-99 of the reference functional full-length human GDE sequence,
wherein said functional truncated GDE polypeptide comprises the sequence of SEQ ID NO:18,
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide comprises or consists of SEQ ID NO: 13, or a functional variant thereof having at least 70% sequence identity with SEQ ID NO:13, such as at least 75% or at least 80% sequence identity, such as at least 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:13.

In a particular embodiment, the functional variant having at least 70% sequence identity with SEQ ID NO: 13, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:13, has substantially the same functionality or enzymatic activity as the GDE polypeptide of SEQ ID NO:13, in particular the same enzymatic activity involved in glycogen degradation in muscular tissues such as heart or quadriceps. In a particular embodiment, the functional variant of SEQ ID NO:13 has substantially the same ability to rescue glycogen accumulation and muscle strength in vivo, as the GDE polypeptide of SEQ ID NO:13. In a particular embodiment, the functional variant of SEQ ID NO: 13 has substantially the same level of expression as the GDE polypeptide of SEQ ID NO: 13.

In a particular embodiment, the functional variant having at least 70% sequence identity with SEQ ID NO: 13, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:13, has the same N-terminal part as SEQ ID NO:13. In a particular embodiment, the most-N terminal amino acids of the functional variant of SEQ ID NO:13 are “MEKSGG” (SEQ ID NO:8). In another particular embodiment, the most-N terminal amino acids of the functional variant of SEQ ID NO:13 correspond to the sequence of SEQ ID NO: 18.

In a particular embodiment, the most N-terminal amino acids of the functional variant of SEQ ID NO:13 may consist of the amino acids 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:13. In other words, according to this embodiment, the functional variant of SEQ ID NO:13 does not comprise any mutation, insertion, or deletion within the region corresponding to amino acids 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:13. In a particular embodiment, the N-terminal end of the functional variant of SEQ ID NO:13 consists of the amino acids 1-50, 1-100, or 1-150 of SEQ ID NO:13. In other words, according to this embodiment, the functional variant of SEQ ID NO: 13 does not comprise any mutation, insertion, or deletion within the region corresponding to amino acids 1-50, 1-100 or 1-150.

In a preferred embodiment, the functional truncated GDE polypeptide comprises or consists of SEQ ID NO: 13.

“Δ1b6” Truncated Polypeptide

In a particular embodiment, the functional truncated GDE polypeptide of the invention comprises a deletion with respect to the reference functional full-length human GDE sequence, wherein said deletion consists of the deletion of amino acids in the N-terminal part of the reference functional full-length human GDE sequence in such a way that the first six amino acids at the N-terminus of the functional truncated GDE polypeptide are “MILRVG” (SEQ ID NO: 9).

In a particular embodiment, the functional truncated GDE polypeptide of the invention comprises a deletion with respect to the reference functional full-length human GDE sequence,
wherein the reference functional full-length human GDE has an amino acid sequence as shown in SEQ ID NO:1 or SEQ ID NO:4, or has an amino acid sequence having at least 80, 85, 90, 95, 96, 97, 98 or at least 99 percent sequence identity to SEQ ID NO:1 or SEQ ID NO:4; and
wherein said deletion consists of the deletion of amino acids in the N-terminal part of the reference functional full-length human GDE sequence in such a way that the first six amino acids at the N-terminus of the functional truncated GDE polypeptide are “MILRVG” (SEQ ID NO: 9).

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-111 of the reference functional full-length GDE sequence, and
wherein the amino acid at position 1 and the amino acid at position 112 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-111 of the reference functional full-length human GDE sequence,
wherein the amino acid at position 1 of the reference functional full-length GDE sequence is not deleted, and
wherein the amino acids from positions 112 to 130, 112 to 150, 112 to 170, 112 to 190, 112 to 210, 112 to 230, 112 to 250, 112 to 270, or the amino acids from positions 112 to 280 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-111 of the reference functional full-length human GDE sequence,
wherein the amino acid at position 1 of the reference functional full-length GDE sequence is not deleted, and
wherein the amino acids from positions 112 to 280 of the reference functional full-length GDE sequence are not deleted.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-111 of the reference functional full-length human GDE sequence,
and wherein said functional truncated GDE polypeptide comprises the sequence “MILRVG” (SEQ ID NO: 9).

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-111 of the reference functional full-length human GDE sequence,
wherein said functional truncated GDE polypeptide comprises the sequence “MILRVG” (SEQ ID NO: 9),
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-111 of the reference functional full-length human GDE sequence,
and wherein said functional truncated GDE polypeptide comprises the sequence of SEQ ID NO:19.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-111 of the reference functional full-length human GDE sequence,
wherein said functional truncated GDE polypeptide comprises the sequence of SEQ ID NO:19,
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide comprises or consists of SEQ ID NO: 14, or a functional variant thereof having at least 70% sequence identity with SEQ ID NO:14, such as at least 75% or at least 80% sequence identity, such as at least 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO: 14.

In a particular embodiment, the functional variant having at least 70% sequence identity with SEQ ID NO: 14, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:14, has substantially the same functionality or enzymatic activity as the GDE polypeptide of SEQ ID NO:14, in particular the same enzymatic activity involved in glycogen degradation in muscular tissues such as heart or quadriceps. In a particular embodiment, the functional variant of SEQ ID NO:14 has substantially the same ability to rescue glycogen accumulation and muscle strength in vivo, as the GDE polypeptide of SEQ ID NO:14. In a particular embodiment, the functional variant of SEQ ID NO:14 has substantially the same level of expression as the GDE polypeptide of SEQ ID NO: 14.

In a particular embodiment, the functional variant having at least 70% sequence identity with SEQ ID NO: 14, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:14, has the same N-terminal part as SEQ ID NO:14. In a particular embodiment, the most-N terminal amino acids of the functional variant of SEQ ID NO:14 are “MILRVG” (SEQ ID NO:9). In another particular embodiment, the most-N terminal amino acids of the functional variant of SEQ ID NO:14 correspond to the sequence of SEQ ID NO:19.

In a further embodiment, the most N-terminal amino acids of the functional variant of SEQ ID NO:14 may consist of the amino acids 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:14. In other words, according to this embodiment, the functional variant of SEQ ID NO:14 does not comprise any mutation, insertion, or deletion within the region corresponding to amino acids 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:14. In a particular embodiment, the N-terminal end of the functional variant of SEQ ID NO: 14 consists of the amino acids 1-50, 1-100, or 1-150 of SEQ ID NO:14. In other words, according to this embodiment, the functional variant of SEQ ID NO: 14 does not comprise any mutation, insertion, or deletion within the region corresponding to amino acids 1-50, 1-100 or 1-150.

In a preferred embodiment, the functional truncated GDE polypeptide comprises or consists of SEQ ID NO: 14.

“Δ1b7” Truncated Polypeptide

In a particular embodiment, the functional truncated GDE polypeptide of the invention comprises a deletion with respect to the reference functional full-length human GDE sequence, wherein said deletion consists of the deletion of amino acids in the N-terminal part of the reference functional full-length human GDE sequence in such a way that the first six amino acids at the N-terminus of the functional truncated GDE polypeptide are “MGADNH” (SEQ ID NO: 10).

In a particular embodiment, the functional truncated GDE polypeptide of the invention comprises a deletion with respect to the reference functional full-length human GDE sequence,
wherein the reference functional full-length human GDE has an amino acid sequence as shown in SEQ ID NO:1 or SEQ ID NO:4, or has an amino acid sequence having at least 80, 85, 90, 95, 96, 97, 98 or at least 99 percent sequence identity to SEQ ID NO:1 or SEQ ID NO:4; and
wherein said deletion consists of the deletion of amino acids in the N-terminal part of the reference functional full-length human GDE sequence in such a way that the first six amino acids at the N-terminus of the functional truncated GDE polypeptide are “MGADNH” (SEQ ID NO: 10).

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-115 of the reference functional full-length GDE sequence, and
wherein the amino acid at position 1 and the amino acid at position 116 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-115 of the reference functional full-length human GDE sequence,
wherein the amino acid at position 1 of the reference functional full-length GDE sequence is not deleted, and
wherein the amino acids from positions 116 to 130, 116 to 150, 116 to 170, 116 to 190, 116 to 210, 116 to 230, 116 to 250, 116 to 270, or the amino acids from positions 116 to 280 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-115 of the reference functional full-length human GDE sequence,
wherein the amino acid at position 1 of the reference functional full-length GDE sequence is not deleted, and
wherein the amino acids from positions 116 to 280 of the reference functional full-length GDE sequence are not deleted.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-115 of the reference functional full-length human GDE sequence,
and wherein said functional truncated GDE polypeptide comprises the sequence “MGADNH” (SEQ ID NO: 10).

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-115 of the reference functional full-length human GDE sequence,
wherein said functional truncated GDE polypeptide comprises the sequence “MGADNH” (SEQ ID NO: 10),
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-115 of the reference functional full-length human GDE sequence,
and wherein said functional truncated GDE polypeptide comprises the sequence of SEQ ID NO:20.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-115 of the reference functional full-length human GDE sequence,
wherein said functional truncated GDE polypeptide comprises the sequence of SEQ ID NO:20,
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide comprises or consists of SEQ ID NO: 15, or a functional variant thereof having at least 70% sequence identity with SEQ ID NO:15, such as at least 75% or at least 80% sequence identity, such as at least 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:15.

In a particular embodiment, the functional variant having at least 70% sequence identity with SEQ ID NO: 15, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:15, has substantially the same functionality or enzymatic activity as the GDE polypeptide of SEQ ID NO:15, in particular the same enzymatic activity involved in glycogen degradation in muscular tissues such as heart or quadriceps. In a particular embodiment, the functional variant of SEQ ID NO:15 has substantially the same ability to rescue glycogen accumulation and muscle strength in vivo, as the GDE polypeptide of SEQ ID NO:15. In a particular embodiment, the functional variant of SEQ ID NO: 15 has substantially the same level of expression as the GDE polypeptide of SEQ ID NO: 15.

In a particular embodiment, the functional variant having at least 70% sequence identity with SEQ ID NO: 15, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:15, has the same N-terminal part as SEQ ID NO:15. In a particular embodiment, the most-N terminal amino acids of the functional variant of SEQ ID NO:15 are “MGADNH” (SEQ ID NO:10). In another particular embodiment, the most-N terminal amino acids of the functional variant of SEQ ID NO: 15 correspond to the sequence of SEQ ID NO:20.

In a further embodiment, the most N-terminal amino acids of the functional variant of SEQ ID NO:15 may consist of the amino acids 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:15. In other words, according to this embodiment, the functional variant of SEQ ID NO:15 does not comprise any mutation, insertion, or deletion within the region corresponding to amino acids 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:15. In a particular embodiment, the N-terminal end of the functional variant of SEQ ID NO:15 consists of the amino acids 1-50, 1-100, or 1-150 of SEQ ID NO:15. In other words, according to this embodiment, the functional variant of SEQ ID NO: 15 does not comprise any mutation, insertion, or deletion within the region corresponding to amino acids 1-50, 1-100 or 1-150.

In a preferred embodiment, the functional truncated GDE polypeptide comprises or consists of SEQ ID NO: 15.

“Δ1b8” Truncated Polypeptide

In a particular embodiment, the functional truncated GDE polypeptide of the invention comprises a deletion with respect to the reference functional full-length human GDE sequence, wherein said deletion consists of the deletion of amino acids in the N-terminal part of the reference functional full-length human GDE sequence in such a way that the first six amino acids at the N-terminus of the functional truncated GDE polypeptide are “MLDCVT” (SEQ ID NO: 11).

In a particular embodiment, the functional truncated GDE polypeptide of the invention comprises a deletion with respect to the reference functional full-length human GDE sequence,
wherein the reference functional full-length human GDE has an amino acid sequence as shown in SEQ ID NO:1 or SEQ ID NO:4, or has an amino acid sequence having at least 80, 85, 90, 95, 96, 97, 98 or at least 99 percent sequence identity to SEQ ID NO:1 or SEQ ID NO:4; and
wherein said deletion consists of the deletion of amino acids in the N-terminal part of the reference functional full-length human GDE sequence in such a way that the first six amino acids at the N-terminus of the functional truncated GDE polypeptide are “MLDCVT” (SEQ ID NO: 11).

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-123 of the reference functional full-length GDE sequence, and
wherein the amino acid at position 1 and the amino acid at position 124 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-123 of the reference functional full-length human GDE sequence,
wherein the amino acid at position 1 of the reference functional full-length GDE sequence is not deleted, and
wherein the amino acids from positions 124 to 130, 124 to 150, 124 to 170, 124 to 190, 124 to 210, 124 to 230, 124 to 250, 124 to 270, or the amino acids from positions 124 to 280 of the reference functional full-length GDE sequence are not deleted.

In a particular embodiment, the functional truncated GDE polypeptide of the invention comprises a deletion with respect to the reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-123 of the reference functional full-length human GDE sequence,
wherein the amino acid at position 1 of the reference functional full-length GDE sequence is not deleted, and
wherein the amino acids from positions 124 to 280 of the reference functional full-length GDE sequence are not deleted.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-123 of the reference functional full-length human GDE sequence,
and wherein said functional truncated GDE polypeptide comprises the sequence “MLDCVT” (SEQ ID NO: 11).

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-123 of the reference functional full-length human GDE sequence,
wherein said functional truncated GDE polypeptide comprises the sequence “MLDCVT” (SEQ ID NO: 11),
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-123 of the reference functional full-length human GDE sequence,
and wherein said functional truncated GDE polypeptide comprises the sequence of SEQ ID NO:21.

In a particular embodiment, the functional truncated GDE polypeptide of the invention comprises a deletion with respect to a reference functional full-length human GDE sequence,
wherein the reference functional full-length human GDE sequence is SEQ ID NO:1 or SEQ ID NO:4, or a functional variant of SEQ ID NO:1 or SEQ ID NO:4 having the same length,
wherein said deletion consists of the deletion of amino acids at positions 2-123 of the reference functional full-length human GDE sequence,
wherein said functional truncated GDE polypeptide comprises the sequence of SEQ ID NO:21,
and wherein the functional truncated GDE polypeptide of the invention comprises at least the amino acid residues at positions 429-666, 866-892, 1088-1194, 1235-1420 with respect to SEQ ID NO:1 or SEQ ID NO:4.

In a particular embodiment, the functional truncated GDE polypeptide comprises or consists of SEQ ID NO: 16, or a functional variant thereof having at least 70% sequence identity with SEQ ID NO:16, such as at least 75% or at least 80% sequence identity, such as at least 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:16.

In a particular embodiment, the functional variant having at least 70% sequence identity with SEQ ID NO: 16, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:16, has substantially the same functionality or enzymatic activity as the GDE polypeptide of SEQ ID NO:16, in particular the same enzymatic activity involved in glycogen degradation in muscular tissues such as heart or quadriceps. In a particular embodiment, the functional variant of SEQ ID NO:16 has substantially the same ability to rescue glycogen accumulation and muscle strength in vivo, as the GDE polypeptide of SEQ ID NO:16. In a particular embodiment, the functional variant of SEQ ID NO:16 has substantially the same level of expression as the GDE polypeptide of SEQ ID NO: 16.

In a particular embodiment, the functional variant having at least 70% sequence identity with SEQ ID NO: 16, such as at least 75% or at least 80% sequence identity, such as 81%, 82%, 83%, 84%, 85%, 86%, 87%, 88%, 89%, 90%, 91%, 92%, 92%, 93%, 94%, 95%, 96%, 97%, 98%, or at least 99% sequence identity with SEQ ID NO:16, has the same N-terminal part as SEQ ID NO:16. In a particular embodiment, the most-N terminal amino acids of the functional variant of SEQ ID NO:16 are “MLDCVT” (SEQ ID NO:11). In another particular embodiment, the most-N terminal amino acids of the functional variant of SEQ ID NO:16 correspond to the sequence of SEQ ID NO:21.

In a further embodiment, the most N-terminal amino acids of the functional variant of SEQ ID NO:16 may consist of the amino acids 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:16. In other words, according to this embodiment, the functional variant of SEQ ID NO:16 does not comprise any mutation, insertion, or deletion within the region corresponding to amino acids 1-10, 1-20, 1-30, 1-40, 1-50, 1-60, 1-70, 1-80, 1-90, 1-100, 1-110, 1-120, 1-130, 1-140, 1-150, 1-175, 1-200 of SEQ ID NO:16. In a particular embodiment, the N-terminal end of the functional variant of SEQ ID NO:16 consists of the amino acids 1-50, 1-100, or 1-150 of SEQ ID NO:16. In other words, according to this embodiment, the functional variant of SEQ ID NO: 16 does not comprise any mutation, insertion, or deletion within the region corresponding to amino acids 1-50, 1-100 or 1-150.

In a preferred embodiment, the functional truncated GDE polypeptide comprises or consists of SEQ ID NO: 16.

Combination with Other Deletions

In a particular embodiment, the functional truncated GDE polypeptide of the invention as described above, which comprises a deletion in the N-terminal part of a reference functional full-length human GDE sequence, may further comprise a deletion or a combination of deletions in other parts of the reference functional full-length human GDE sequence.

In a particular embodiment, the functional truncated GDE polypeptide comprises:
one and only one deletion of consecutive amino acids in the N-terminal part of the reference functional full-length human GDE sequence, wherein the N-terminal part corresponds to the first 280 amino acid residues of the reference functional full length human GDE sequence,
another deletion or a combination of deletions in other parts of the reference functional full-length human GDE sequence.

In a particular embodiment, the functional truncated GDE polypeptide comprises:
one and only one deletion of consecutive amino acids in the N-terminal part of the reference functional full-length human GDE sequence, wherein the N-terminal part corresponds to the first 280 amino acid residues of the reference functional full length human GDE sequence, and
another deletion or a combination of deletions in other parts of the reference functional full-length human GDE sequence,
   wherein the reference functional full-length human GDE sequence is selected from the group consisting of SEQ ID NO:1 to SEQ ID NO:6,
   and wherein the deletion is referred to as Δ1b4, Δ1b5, Δlb6, Δ1b7, 41b8 in table 1.

In a particular embodiment, the functional truncated GDE polypeptide of the invention comprises at least the amino acid residues at positions 429-666, 866-892, 1088-1194, 1235-1420 with respect to SEQ ID NO: 1 or SEQ ID NO:4.

In a particular embodiment, the functional truncated GDE polypeptide of the invention, which comprises a deletion in the N-terminal part of a reference functional full-length human GDE sequence, may further comprise a deletion or a combination of deletions in the C-terminal part of the reference full-length GDE sequence, and/or a in the central domain of the reference full-length GDE sequence.

In a particular embodiment, the C-terminal part of the reference full length GDE sequence corresponds to a region consisting of the last 112 amino acids, i.e. consisting of the most C-terminal 112 amino acids of said reference full-length human GDE sequence.

In a particular embodiment, the central domain of the reference full-length GDE sequence of SEQ ID NO: 1 or SEQ ID NO:4, corresponds to a region consisting of amino acids at positions 710 to 865 of SEQ ID NO:1 or SEQ ID NO: 4.

In a particular embodiment, the functional truncated GDE polypeptide comprises:
one and only one deletion in the N-terminal part of the reference functional full-length human GDE sequence, wherein the N-terminal part corresponds to a region consisting of the first 280 amino acid residues of the reference functional full length human GDE sequence, and
another deletion or combination of deletions in other parts of the reference functional full-length human GDE sequence,
   wherein the reference functional full-length human GDE sequence is selected from the group consisting of SEQ ID NO:1 to SEQ ID NO:6,
   and wherein the deletion in the N-terminal part is referred to as Δ1b4, Δ1b5, Δlb6, Δ1b7, Δ1b8 in table 1;
   and wherein the addition deletion or combination of deletions in other parts of the reference functional full-length human GDE sequence is (are) selected from any deletion referred to as Δ1, Δ2, Δ3, Δ4, Δ5, Δ6, and Δ7 in table 2:

TABLE 2

| | Position of the | Position of the | Position of the |
|---|---|---|---|
| | deleted amino | deleted amino | deleted amino |
| | acids with respect | acids with respect | acids with respect |
| | to SEQ ID NO: 1 | to SEQ ID NO: 2 | to SEQ ID NO: 3 |
| Deletion | or SEQ ID NO: 4 | or SEQ ID NO: 5 | or SEQ ID NO: 6 |
| Δ1 | 361-428 | 344-411 | 345-412 |
| Δ2 | 668-769 | 651-752 | 652-753 |
| Δ3 | 895-1087 | 878-1070 | 879-1071 |
| Δ4 | 223-320 | 206-303 | 207-304 |
| Δ5 | 360-428 | 343-411 | 344-412 |
| Δ6 | 669-720 | 652-703 | 653-704 |
| Δ7 | 1421-1532 | 1404-1515 | 1405-1516 |

### 2-Nucleic Acid Molecule Encoding the N-Terminal Truncated GDE Polypeptide

In another aspect, the invention relates to a nucleic acid molecule encoding the functional truncated GDE polypeptide as defined above.

The term “nucleic acid molecule” (or nucleic acid sequence) refers to a DNA or RNA molecule in single or double stranded form, particularly a DNA encoding a functional truncated GDE polypeptide according to the invention.

In a preferred embodiment, the nucleic acid molecule encoding the functional truncated GDE polypeptide is small enough to be packaged in combination with appropriate regulatory sequences, into a gene therapy vector, such as an AAV vector. The size of the nucleic acid molecule encoding the functional truncated GDE polypeptide is preferably less than about 4.5 kb, preferably less than about 4.4 kb.

By “gene therapy vector” is meant any vector suitable for gene therapy. In particular, the gene therapy vector may be a plasmid or a recombinant virus such as a viral vector derived from a retrovirus or a lentivirus. Preferably, the viral vector is an AAV vector, such as an AAV vector suitable for transducing liver tissues or muscle cells. Extensive experience in clinical trials and in preclinical model of muscle diseases indicates adeno-associated virus (AAV) as the vector of choice for in vivo gene therapy for GSDIII. These vectors efficiently transduce liver and muscle, their production is scalable and compared to other gene therapy vectors they have a relatively low immunogenicity profile. However, one of the biggest limitations in the use of AAV for gene replacement is their limited encapsidation size limit (about 5 kb). Indeed, during recombinant AAV production, genomes larger than 5 kb are encapsidated with low efficacy and the resulting AAV may contain fragmented genomes reducing the efficacy of gene transfer.

The sequence of the nucleic acid molecule of the invention, encoding the functional truncated GDE polypeptide may be optimized for expression of the GDE polypeptide in vivo. Sequence optimization may include a number of changes in a nucleic acid sequence, including codon optimization, increase of GC content, decrease of the number of CpG islands, decrease of the number of alternative open reading frames (ARFs) and/or decrease of the number of splice donor and splice acceptor sites. Because of the degeneracy of the genetic code, different nucleic acid molecules may encode the same protein. It is also well known that the genetic codes of different organisms are often biased towards using one of the several codons that encode the same amino acid over the others. Through codon optimization, changes are introduced in a nucleotide sequence that take advantage of the codon bias existing in a given cellular context so that the resulting codon optimized nucleotide sequence is more likely to be expressed in such given cellular context at a relatively high level compared to the non-codon optimized sequence. In a preferred embodiment of the invention, such optimized nucleotide sequence encoding a functional truncated GDE polypeptide is codon-optimized to improve its expression in human cells compared to non-codon optimized nucleotide sequences coding for the same functional truncated GDE polypeptide, for example by taking advantage of the human specific codon usage bias. The nucleic acid sequence encoding full-length human GDE isoform 1 is as shown in SEQ ID NO:32. An example of corresponding codon optimized sequence is as shown in SEQ ID NO:33.

In a particular embodiment, the nucleic acid molecule of the invention encodes the functional truncated GDE polypeptide having the amino acid sequence shown in SEQ ID NO:12. In a particular embodiment, the nucleic acid molecule of the invention comprises or consists of the sequence shown in SEQ ID NO: 22 or SEQ ID NO:23, encoding the functional truncated GDE polypeptide having the amino acid sequence shown in SEQ ID NO:12. In a particular embodiment, the nucleic acid molecule encoding the functional truncated GDE polypeptide as defined above has at least 80, at least 85, at least 90 or at least 95 percent identity to the nucleotide sequence of SEQ ID NO:22 or SEQ ID NO:23, preferably SEQ ID NO: 22. In a particular embodiment, the nucleic acid molecule of the invention has at least 95 percent identity, for example at least 96, 97, 98, 99 or 100 percent identity to the nucleotide sequence of SEQ ID NO: 22 or SEQ ID NO:23, preferably SEQ ID NO:22.

In a particular embodiment, the nucleic acid molecule of the invention encodes the functional truncated GDE polypeptide having the amino acid sequence shown in SEQ ID NO:13. In a particular embodiment, the nucleic acid molecule of the invention comprises or consists of the sequence shown in SEQ ID NO: 24 or SEQ ID NO:25 encoding the functional truncated GDE polypeptide having the amino acid sequence shown in SEQ ID NO:13. In a particular embodiment, the nucleic acid molecule encoding the functional truncated GDE polypeptide as defined above has at least 80, at least 85, at least 90 or at least 95 percent identity to the nucleotide sequence of SEQ ID NO:24 or SEQ ID NO:25, preferably SEQ ID NO: 24. In a particular embodiment, the nucleic acid molecule of the invention has at least 95 percent identity, for example at least 96, 97, 98, 99 or 100 percent identity to the nucleotide sequence of SEQ ID NO: 24 or SEQ ID NO:25, preferably SEQ ID NO:24.

In a particular embodiment, the nucleic acid molecule of the invention encodes the functional truncated GDE polypeptide having the amino acid sequence shown in SEQ ID NO:14. In a particular embodiment, the nucleic acid molecule of the invention comprises or consists of the sequence shown in SEQ ID NO: 26 or SEQ ID NO:27 encoding the functional truncated GDE polypeptide having the amino acid sequence shown in SEQ ID NO:14. In a particular embodiment, the nucleic acid molecule encoding the functional truncated GDE polypeptide as defined above has at least 80, at least 85, at least 90 or at least 95 percent identity to the nucleotide sequence of SEQ ID NO:26 or SEQ ID NO:27, preferably SEQ ID NO: 26. In a particular embodiment, the nucleic acid molecule of the invention has at least 95 percent identity, for example at least 96, 97, 98, 99 or 100 percent identity to the nucleotide sequence of SEQ ID NO: 26 or SEQ ID NO:27, preferably SEQ ID NO:26.

In a particular embodiment, the nucleic acid molecule of the invention encodes the functional truncated GDE polypeptide having the amino acid sequence shown in SEQ ID NO:15. In a particular embodiment, the nucleic acid molecule of the invention comprises or consists of the sequence shown in SEQ ID NO: 28 or SEQ ID NO:29 encoding the functional truncated GDE polypeptide having the amino acid sequence shown in SEQ ID NO:15. In a particular embodiment, the nucleic acid molecule encoding the functional truncated GDE polypeptide as defined above has at least 80, at least 85, at least 90 or at least 95 percent identity to the nucleotide sequence of SEQ ID NO:28 or SEQ ID NO:29, preferably SEQ ID NO: 28. In a particular embodiment, the nucleic acid molecule of the invention has at least 95 percent identity, for example at least 96, 97, 98, 99 or 100 percent identity to the nucleotide sequence of SEQ ID NO: 28 or SEQ ID NO:29, preferably SEQ ID NO:28.

In a particular embodiment, the nucleic acid molecule of the invention encodes the functional truncated GDE polypeptide having the amino acid sequence shown in SEQ ID NO:16. In a particular embodiment, the nucleic acid molecule of the invention comprises or consists of the sequence shown in SEQ ID NO: 30 or SEQ ID NO:31 encoding the functional truncated GDE polypeptide having the amino acid sequence shown in SEQ ID NO:16. In a particular embodiment, the nucleic acid molecule encoding the functional truncated GDE polypeptide as defined above has at least 80, at least 85, at least 90 or at least 95 percent identity to the nucleotide sequence of SEQ ID NO:30 or SEQ ID NO:31, preferably SEQ ID NO: 30. In a particular embodiment, the nucleic acid molecule of the invention has at least 95 percent identity, for example at least 96, 97, 98, 99 or 100 percent identity to the nucleotide sequence of SEQ ID NO: 30 or SEQ ID NO:31, preferably SEQ ID NO:30.

The nucleic acid molecule encoding the functional truncated GDE polypeptide as defined above may have at least 80, at least 85, at least 90 or at least 95 percent identity to any of the nucleotide sequences of SEQ ID NO:22 to 31. In a particular embodiment, the nucleic acid molecule of the invention has at least 95 percent identity, for example at least 96, 97, 98, 99 or 100 percent identity to any of the nucleotide sequences of SEQ ID NO:22 to 31.

Preferably, the nucleic acid molecule encoding the functional truncated GDE polypeptide as defined above may have at least 80, at least 85, at least 90 or at least 95 percent identity to any of the nucleotide sequences of SEQ ID NO:22, SEQ ID NO:24, SEQ ID NO:26, SEQ ID NO:28 or SEQ ID NO:30. In a particular embodiment, the nucleic acid molecule of the invention has at least 95 percent identity, for example at least 96, 97, 98, 99 or 100 percent identity to any of the nucleotide sequences of SEQ ID NO: 22, SEQ ID NO:24, SEQ ID NO:26, SEQ ID NO:28 or SEQ ID NO:30.

### 3-Nucleic Acid Construct

The invention also relates to a nucleic acid construct comprising a nucleic acid molecule of the invention as described above. The nucleic acid construct may correspond to an expression cassette comprising the nucleic acid sequence of the invention, operably linked to one or more expression control sequences and/or other sequences improving the expression. As used herein, the term “operably linked” refers to a linkage of polynucleotide elements in a functional relationship. A nucleic acid is “operably linked” when it is placed into a functional relationship with another nucleic acid sequence. For instance, a promoter, or another transcription regulatory sequence, is operably linked to a coding sequence if it affects the transcription of the coding sequence. Such expression control sequences are known in the art, such as promoters, enhancers (such as cis-regulatory modules (CRMs)), introns, polyA signals, etc.

In a particular embodiment, the expression cassette may include a promoter. The promoter may be a ubiquitous or tissue-specific promoter, in particular a promoter able to promote expression in cells or tissues in which expression of GDE is desirable such as in cells or tissues in which GDE expression is desirable in GDE-deficient patients. Preferably, the promoter is a ubiquitous promoter.

In a first particular embodiment, the promoter is a muscle-specific promoter. Non-limiting examples of muscle-specific promoters include the muscle creatine kinase (MCK) promoter. Non-limiting examples of suitable muscle creatine kinase promoters are human muscle creatine kinase promoters and truncated murine muscle creatine kinase [(tMCK) promoters] (Wang B et al, Construction and analysis of compact muscle-selective promoters for AAV vectors. Gene Ther. 2008 November; 15 (22): 1489-99) (representative GenBank Accession No. AF188002). Human muscle creatine kinase has the Gene ID No. 1158 (representative GenBank Accession No. NC_000019.9, accessed on Dec. 26, 2012). Other examples of muscle-specific promoters include a synthetic promoter C5.12 (spC5.12, alternatively referred to herein as “C5.12”), such as the spC5.12 or the spC5.12 promoter (disclosed in Wang et al., Gene Therapy volume 15, pages 1489-1499 (2008)), the MHCK7 promoter (Salva et al. Mol Ther. 2007 February; 15 (2): 320-9), myosin light chain (MLC) promoters, for example MLC2 (Gene ID No. 4633; representative GenBank Accession No. NG_007554.1, accessed on Dec. 26, 2012); myosin heavy chain (MHC) promoters, for example alpha-MHC (Gene ID No. 4624; representative GenBank Accession No. NG_023444.1, accessed on Dec. 26, 2012); desmin promoters (Gene ID No. 1674; representative GenBank Accession No. NG_008043.1, accessed on Dec. 26, 2012); cardiac troponin C promoters (Gene ID No. 7134; representative GenBank Accession No. NG_008963.1, accessed on Dec. 26, 2012); troponin I promoters (Gene ID Nos. 7135, 7136, and 7137; representative GenBank Accession Nos. NG_016649.1, NG_011621.1, and NG_007866.2, accessed on Dec. 26, 2012); myoD gene family promoters (Weintraub et al., Science, 251, 761 (1991); Gene ID No. 4654; representative GenBank Accession No. NM_002478, accessed on Dec. 26, 2012); alpha actin promoters (Gene ID Nos. 58, 59, and 70; representative GenBank Accession Nos. NG_006672.1, NG_011541.1, and NG_007553.1, accessed on Dec. 26, 2012); beta actin promoters (Gene ID No. 60; representative GenBank Accession No. NG_007992.1, accessed on Dec. 26, 2012); gamma actin promoters (Gene ID No. 71 and 72; representative GenBank Accession No. NG_011433.1 and NM_001199893, accessed on Dec. 26, 2012); muscle-specific promoters residing within intron 1 of the ocular form of Pitx3 (Gene ID No. 5309) (Coulon et al; the muscle-selective promoter corresponds to residues 11219-11527 of representative GenBank Accession No. NG_008147, accessed on Dec. 26, 2012); and the promoters described in US Patent Publication US 2003/0157064, and CK6 promoters (Wang et al 2008 doi: 10.1038/gt. 2008.104). In another particular embodiment, the muscle-specific promoter is the E-Syn promoter described in Wang et al., Gene Therapy volume 15, pages 1489-1499 (2008), comprising the combination of a MCK-derived enhancer and of the spC5.12 promoter. In a particular embodiment of the invention, the muscle-specific promoter is selected in the group consisting of a spC5.12 promoter, the MHCK7 promoter, the E-syn promoter, a muscle creatine kinase myosin light chain (MLC) promoter, a myosin heavy chain (MHC) promoter, a cardiac troponin C promoter, a troponin I promoter, a myoD gene family promoter, an alpha actin promoter, an beta actin promoter, an gamma actin promoter, a muscle-specific promoter residing within intron 1 of the ocular form of Pitx3, a CK6 promoter, a CK8 promoter and an Acta1 promoter. In a further embodiment, the muscle-specific promoter is selected in the group consisting of the spC5.12, desmin and MCK promoters. In a further embodiment, the muscle-specific promoter is selected in the group consisting of the spC5.12 and MCK promoters. In a preferred embodiment, the muscle-specific promoter is the spC5.12 promoter.

In another particular embodiment, the promoter is a liver-specific promoter. Non-limiting examples of liver-specific promoters include the HSE promoter (Hepatic Specific Promoter), the alpha-1 antitrypsin promoter (hAAT), the transthyretin promoter, the albumin promoter, the thyroxine-binding globulin (TBG) promoter, the LSP promoter (comprising a thyroid hormone-binding globulin promoter sequence, two copies of an alpha1-microglobulin/bikunin enhancer sequence, and a leader sequence-Ill, C. R., et al. (1997). Optimization of the human factor VIII complementary DNA expression plasmid for gene therapy of hemophilia A. Blood Coag. Fibrinol. 8: S23-S30.), etc. Other useful liver-specific promoters are known in the art, for example those listed in the Liver Specific Gene Promoter Database compiled the Cold Spring Harbor Laboratory (http://rulai.cshl.edu/LSPD/). Preferred liver-specific promoters in the context of the invention are promoters comprising a H1 enhancer and a TTR promoter. One example of such liver-specific promoter is the HSE promoter. The HSE promoter contains a mouse TTR promoter and the H1 enhancer described in SEQ NO: 51, and is derived from the initial paper of Robert H. Costa et al. 1986 (Transcriptional control of the mouse prealbumin (transthyretin) gene: both promoter sequences and a distinct enhancer are cell specific. Mol Cell Biol. 1986; 6 (12): 4697-4708). In a preferred embodiment, the liver specific promoter is the HSE promoter having the sequence SEQ ID NO: 53, or a functional variant thereof having at least 80% sequence identity to SEQ ID NO:53, such as at least 85%, in particular at least 90%, more particularly at least 91%, 92%, 93%, 94%, 95%, 96%, 97%, 98% or even at least 99% sequence identity to SEQ ID NO:53.

In another particular embodiment, the promoter is a neuron-specific promoter. Non-limiting examples of neuron-specific promoters include, but are not limited to the following: synapsin-1 (Syn) promoter, neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al. Neuron, 15:373-84 (1995)), among others which will be apparent to the skilled artisan. In a particular embodiment, the neuron-specific promoter is the Syn promoter. Other neuron-specific promoters include, without limitation: synapsin-2 promoter, tyrosine hydroxylase promoter, dopamine β-hydroxylase promoter, hypoxanthine phosphoribosyltransferase promoter, low affinity NGF receptor promoter, and choline acetyl transferase promoter (Bejanin et al., 1992; Carroll et al., 1995; Chin and Greengard, 1994; Foss-Petter et al., 1990; Harrington et al., 1987; Mercer et al., 1991; Patei et al., 1986). Representative promoters specific for the motor neurons include, without limitation, the promoter of the Calcitonin Gene-Related Peptide (CGRP), a known motor neuron-derived factor. Other promoters functional in motor neurons include the promoters of Choline Acetyl Transferase (ChAT), Neuron Specific Enolase (NSE), Synapsin and Hb9. Other neuron-specific promoters useful in the present invention include, without limitation: GFAP (for astrocytes), Calbindin 2 (for interneurons), Mnx1 (motorneurons), Nestin (neurons), Parvalbumin, Somatostation and Plp1 (oligodendrocytes and Schwann cells).

In a preferred embodiment, the promoter is a ubiquitous promoter. Representative ubiquitous promoters include the cytomegalovirus enhancer/chicken beta actin (CAG) promoter, the cytomegalovirus enhancer/promoter (CMV) (optionally with the CMV enhancer) [see, e.g., Boshart et al, Cell, 41:521-530 (1985)] or a short version of the CMV promoter, the PGK promoter, the SV40 early promoter, the retroviral Rous sarcoma virus (RSV) LTR promoter (optionally with the RSV enhancer), the dihydrofolate reductase promoter, the β-actin promoter, the phosphoglycerol kinase (PGK) promoter, the EF1 alpha (EFla) promoter or a short version of the EFla promoter, and the Ins84 promoter (as described in WO2020/219949). In a preferred embodiment, the promoter is a truncated version of the CMV promoter.

In addition, the promoter may also be an endogenous promoter such as the albumin promoter or the GDE promoter.

Short promoters are of particular interest in this invention, such as shorter versions of known promoters. In a preferred embodiment, the promoter is less than about 500pb, preferably less than about 450pb, preferably less than about 400pb.

In a preferred embodiment, the promoter is a shorter version of any promoter as herein described, such as a shorter version of the CMV promoter or a shorter version of the EFla promoter or a promoter comprising a H1 enhancer and a TTR promoter.

In a preferred embodiment, the promoter is a short version of the CMV promoter. More preferably, the promoter is a short version of the CMV promoter having the sequence SEQ ID NO:43, or a functional variant thereof having at least 80% sequence identity to SEQ ID NO:43, such as at least 85%, in particular at least 90%, more particularly at least 91%, 92%, 93%, 94%, 95%, 96%, 97%, 98% or even at least 99% sequence identity to SEQ ID NO:43.

In another particular embodiment, the promoter is a short version of the EFla promoter. Preferably, the promoter is a short version of the EFla promoter having the sequence as shown in SEQ ID NO:52, or a functional variant thereof having at least 80% sequence identity to SEQ ID NO:52, such as at least 85%, in particular at least 90%, more particularly at least 91%, 92%, 93%, 94%, 95%, 96%, 97%, 98% or even at least 99% sequence identity to SEQ ID NO:52.

In another particular embodiment, the promoter is the Ins84 promoter as described in WO2020/219949.

The expression cassette comprising the nucleic acid molecule of the invention may be adapted, depending on the target population of GSDIII patient, depending on the clinical manifestations of the GSDIII disease and/or depending on the target tissue. For example, for patients having clinical manifestations of GSDIII disease predominantly in muscles (such as adolescents or adults GSDIII patients), the expression cassette preferably comprises a muscle-specific promoter or any promoter able to induce a strong expression of the nucleic acid molecule of the invention in muscles, such as the miniCMV promoter as described above, in particular the miniCMV promoter of SEQ ID NO:43. For patients having clinical manifestations of GSDIII disease in the liver, the expression cassette preferably comprises a liver-specific promoter or any promoter able to induce a strong expression of the nucleic acid molecule of the invention in the liver, such as the HSE promoter as described above, in particular the HSE promoter of SEQ ID NO:. 53. For patients having clinical manifestation of GSDIII disease in muscles and in the liver, a promoter able to induce the expression of the nucleic acid molecule of the invention in both tissues is preferably used.

In a particular embodiment, the promoter is associated to an enhancer sequence, such as a cis-regulatory module (CRMs) or an artificial enhancer sequence. CRMs useful in the practice of the present invention include those described in Rincon et al., Mol Ther. 2015 January; 23 (1): 43-52, Chuah et al., Mol Ther. 2014 September; 22 (9): 1605-13 or Nair et al., Blood. 2014 May 15;123 (20): 3195-9. Other regulatory elements that are, in particular, able to enhance muscle-specific expression of genes, in particular expression in cardiac muscle and/or skeletal muscle, are those disclosed in WO2015110449. Particular examples of nucleic acid regulatory elements that comprise an artificial sequence include the regulatory elements that are obtained by rearranging the transcription factor binding sites (TFBS) that are present in the sequences disclosed in WO2015110449. Said rearrangement may encompass changing the order of the TFBSs and/or changing the position of one or more TFBSs relative to the other TFBSs and/or changing the copy number of one or more of the TFBSs. For example, a nucleic acid regulatory element for enhancing muscle-specific gene expression, in particular cardiac and skeletal muscle-specific gene expression, may comprise binding sites for E2A, HNH 1, NF1, C/EBP, LRF, MyoD, and SREBP; or for E2A, NF1, p53, C/EBP, LRF, and SREBP; or for E2A, HNH 1, HNF3a, HNF3b, NF1, C/EBP, LRF, MyoD, and SREBP; or E2A, HNF3a, NF1, C/EBP, LRF, MyoD, and SREBP; or for E2A, HNF3a, NF1, CEBP, LRF, MyoD, and SREBP; or for HNF4, NF1, RSRFC4, C/EBP, LRF, and MyoD, or NF1, PPAR, p53, C/EBP, LRF, and MyoD. For example, a nucleic acid regulatory element for enhancing muscle-specific gene expression, in particular skeletal muscle-specific gene expression, may also comprise binding sites for E2A, NF1, SRFC, p53, C/EBP, LRF, and MyoD; or for E2A, NF1, C/EBP, LRF, MyoD, and SREBP; or for E2A, HNF3a, C/EBP, LRF, MyoD, SEREBP, and Tall_b; or for E2A, SRF, p53, C/EBP, LRF, MyoD, and SREBP; or for HNF4, NF1, RSRFC4, C/EBP, LRF, and SREBP; or for E2A, HNF3a, HNF3b, NF1, SRF, C/EBP, LRF, MyoD, and SREBP; or for E2A, CEBP, and MyoD. In further examples, these nucleic acid regulatory elements comprise at least two, such as 2, 3, 4, or more copies of one or more of the TFBSs recited before. Other regulatory elements that are, in particular, able to enhance liver-specific expression of genes, are those disclosed in WO2009130208.

In a particular embodiment, the enhancer is a short-sized enhancer. In particular, an enhancer for use in the invention may consist of 10 to 175 nucleotides, such as 40 to 100 nucleotides, in particular 50 to 80 nucleotides. In a particular embodiment, the enhancer is the 72 nucleotide HS-CRM8 enhancer consisting of SEQ ID NO:51, or a functional variant of SEQ ID NO:51 having an enhancer activity. In another embodiment, the enhancer is a functional variant of the 72 nucleotide HS-CRM8 enhancer that is at least 80% identical to SEQ ID NO:51, such as at least 85% identical, in particular at least 90% identical, more particularly at least 91%, 92%, 93%, 94%, 95%, 96%, 97%, 98% or even at least 99% identical to SEQ ID NO:51.

In another particular embodiment, the nucleic acid construct comprises an intron, in particular an intron placed between the promoter and the GDE coding sequence. An intron may be introduced to increase mRNA stability and the production of the protein. In a further embodiment, the intron is a human beta globin b2 (or HBB2) intron, a coagulation factor IX (FIX) intron, a SV40 intron, a hCMV intron A (hCMVI), a TPL intron (TPLI), a CHEF1 gene intron1 (CHEF1), a MVM intron (Wu et al, 2008), a FIX truncated intron 1 (Wu et al., 2008, Mol Ther, 16 (2): 280-289; Kurachi et al., 1995, J Biol Chem., 270 (10): 5276-5281), a β-globin/immunoglobin heavy chain hybrid intron (5′-donor site from a human β-globin intron and 3′-acceptor site from an immunoglobulin heavy chain variable region intron, Wu et al., 2008, Mol Ther, 16 (2): 280-289; Kurachi et al., 1995, J Biol Chem., 270 (10): 5276-5281), a hybrid intron consisting of an adenovirus splice donor and an immunoglobulin G splice (Wong et al., 1985, Chromosoma, 92 (2): 124-135; Yew et al., 1997, Hum Gene Ther, 8 (5): 575-584; Choi T. et al., 1991, Mol Cell Biol, 11 (6): 3070-3074; Huang et al., 1990, Mol Cell Biol., 10 (4): 1805-1810), a hybrid 19S/16S SV40 intron (5′-donor site from 19S intron and 3′-acceptor site from 16S intron, Yew et al., 1997, Hum Gene Ther, 8 (5): 575-584) or a chicken beta-globin intron. In another further embodiment, the intron is a modified intron (in particular a modified HBB2 or FIX intron) designed to decrease the number of, or even totally remove, alternative open reading frames (ARFs) found in said intron. Preferably, ARFs are removed whose length spans over 50 bp and have a stop codon in frame with a start codon. ARFs may be removed by modifying the sequence of the intron. For example, modification may be carried out by way of nucleotide substitution, insertion or deletion, preferably by nucleotide substitution. As an illustration, one or more nucleotides, in particular one nucleotide, in an ATG or GTG start codon present in the sequence of the intron of interest may be replaced resulting in a non-start codon. For example, an ATG or a GTG may be replaced by a CTG, which is not a start codon, within the sequence of the intron of interest.

The classical HBB2 intron is shown in SEQ ID NO:34. For example, this HBB2 intron may be modified by eliminating start codons (ATG and GTG codons) within said intron. In a particular embodiment, the modified HBB2 intron has the sequence shown in SEQ ID NO:35. The classical FIX intron is derived from the first intron of human FIX and is shown in SEQ ID NO:36. FIX intron may be modified by eliminating start codons (ATG and GTG codons) within said intron. In a particular embodiment, the modified FIX intron has the sequence shown in SEQ ID NO:37. The classical chicken-beta globin intron used in nucleic acid constructs is shown in SEQ ID NO:38. Chicken-beta globin intron may be modified by eliminating start codons (ATG and GTG codons) within said intron. In a particular embodiment, the modified chicken-beta globin intron has the sequence shown in SEQ ID NO:39.

The inventors have previously shown in WO2015/162302 that such a modified intron, in particular a modified HBB2 or FIX intron, has advantageous properties and can significantly improve the expression of a transgene.

In a particular embodiment, the nucleic acid construct of the invention is an expression cassette comprising, in the 5′ to 3′ orientation, a promoter optionally preceded by an enhancer, the coding sequence of the invention (i.e. the nucleic acid molecule encoding the functional truncated GDE polypeptide), and a polyadenylation signal such as the pA58 polyadenylation signal (pA58 polyA), the bovine growth hormone polyadenylation signal (bGH polyA), the SV40 polyadenylation signal, or another naturally occurring or artificial polyadenylation signal. Preferably, the polyadenylation signal is the bGH polyA or the pA58 polyA, more preferably the pA58 polyA. In a particular embodiment, the polyadenylation signal is the bGH poly A as shown in SEQ ID NO:41. In a particular embodiment, a very short polyA signal is used. For example, a very short polyA signal comprising less than 20 nucleotides is used. In a particular embodiment, the polyadenylation signal is the human soluble neuropilin-1 (sNRP) polyadenylation signal (sNRP polyA; SEQ ID NO:40). In a preferred embodiment, the polyadenylation signal is the pA58 polyadenylation signal as shown in SEQ ID NO:42.

In a particular embodiment, the nucleic acid construct of the invention is an expression cassette comprising, in the 5′ to 3′ orientation, a promoter optionally preceded by an enhancer, an intron, the coding sequence of the invention, and a polyadenylation signal. In another embodiment, the nucleic acid construct of the invention is an expression cassette comprising, in the 5′ to 3′ orientation, a promoter, the coding sequence of the invention, and a polyadenylation signal. In another embodiment, the nucleic acid construct of the invention is an expression cassette comprising, in the 5′ to 3′ orientation, an enhancer, a promoter, the coding sequence of the invention, and a polyadenylation signal. In a further particular embodiment, the nucleic acid construct of the invention is an expression cassette comprising, in the 5′ to 3′ orientation, an enhancer, a promoter, an intron, the coding sequence of the invention, and a polyadenylation signal. In a further particular embodiment of the invention the expression cassette comprising, in the 5′ to 3′ orientation a promoter, an optional intron, the coding sequence of the invention and a polyA signal.

In another embodiment, the nucleic acid construct of the invention is an expression cassette comprising, in the 5′ to 3′ orientation, a SpC5-12 promoter or a CMV promoter such as a mini-CMV promoter, the coding sequence of the invention, and a polyadenylation signal (such as a bGH polyA or a pA58 polyA, in particular a pA58 polyA). In another embodiment, the nucleic acid construct of the invention is an expression cassette comprising, in the 5′ to 3′ orientation, an enhancer, a SpC5-12 promoter or a CMV promoter such as a mini-CMV promoter, the coding sequence of the invention, and a polyadenylation signal (such as a bGH polyA or a pA58 polyA, in particular a pA58 polyA).

In a further particular embodiment, the expression cassette comprises, in the 5′ to 3′ orientation: a SpC5-12 promoter; a sequence coding the amino acid sequence of SEQ ID NO:12, SEQ ID NO:13, SEQ ID NO: 14, SEQ ID NO:15 or SEQ ID NO:16; and a bGH polyA or pA58 polyA, in particular a pA58 polyA. In another embodiment, the expression cassette comprises, in the 5′ to 3′ orientation: a CMV promoter; a sequence coding the amino acid sequence of SEQ ID NO:12, SEQ ID NO:13, SEQ ID NO: 14, SEQ ID NO:15 or SEQ ID NO:16; and a bGH polyA or pA58 polyA, in particular a pA58 polyA.

In a preferred embodiment, the expression cassette comprises in the 5′ to 3′ orientation: a CMV promoter such as a mini-CMV promoter, in particular the mini-CMV promoter of SEQ ID NO:43; a sequence encoding the functional truncated GDE polypeptide as defined above such as a sequence coding the amino acid sequence of SEQ ID NO:12, SEQ ID NO:13, SEQ ID NO:14, SEQ ID NO:15 or SEQ ID NO:16; and a bGH polyA or pA58 polyA, in particular a pA58 polyA.

In a particular embodiment, the expression cassette comprises in the 5′ to 3′ orientation: a CMV promoter such as a mini-CMV promoter, in particular the mini-CMV promoter of SEQ ID NO:43; a sequence encoding the functional truncated GDE polypeptide as defined above, such as a sequence selected in the group consisting of SEQ ID NO: 22 to SEQ ID NO:31; and a bGH polyA or pA58 polyA, in particular a pA58 polyA.

In a particular embodiment, the expression cassette comprises in the 5′ to 3′ orientation: a CMV promoter such as a mini-CMV promoter, in particular the mini-CMV promoter of SEQ ID NO:43; a sequence encoding the functional truncated GDE polypeptide of SEQ ID NO: 12; and a bGH polyA or pA58 polyA, in particular a pA58 polyA. In a particular embodiment, the expression cassette comprises in the 5′ to 3′ orientation: a CMV promoter such as a mini-CMV promoter, in particular the mini-CMV promoter of SEQ ID NO:43; the sequence of SEQ ID NO: 22 or SEQ ID NO: 23 encoding the functional truncated GDE polypeptide of SEQ ID NO: 12; and a bGH polyA or pA58 polyA, in particular a pA58 polyA. In a particular embodiment, the expression cassette comprises in the 5′ to 3′ orientation: the mini-CMV promoter of SEQ ID NO:43; the sequence of SEQ ID NO: 22 or SEQ ID NO: 23 encoding the functional truncated GDE polypeptide of SEQ ID NO: 12; and the pA58 polyA of SEQ ID NO:42.

In a preferred embodiment, the expression cassette comprises in the 5′ to 3′ orientation: a CMV promoter such as a mini-CMV promoter, in particular the mini-CMV promoter of SEQ ID NO:43; a sequence encoding the functional truncated GDE polypeptide of SEQ ID NO: 13; and a bGH polyA or pA58 polyA, in particular a pA58 polyA. In a particular embodiment, the expression cassette comprises in the 5′ to 3′ orientation: a CMV promoter such as a mini-CMV promoter, in particular the mini-CMV promoter of SEQ ID NO:43; the sequence of SEQ ID NO: 24 or SEQ ID NO: 25 encoding the functional truncated GDE polypeptide of SEQ ID NO: 13; and a bGH polyA or pA58 polyA, in particular a pA58 polyA. In a particular embodiment, the expression cassette comprises in the 5′ to 3′ orientation: the mini-CMV promoter of SEQ ID NO:43; the sequence of SEQ ID NO: 24 or SEQ ID NO: 25 encoding the functional truncated GDE polypeptide of SEQ ID NO: 13; and the pA58 polyA of SEQ ID NO:42.

In a further embodiment, the expression cassette comprises in the 5′ to 3′ orientation: a CMV promoter such as a mini-CMV promoter, in particular the mini-CMV promoter of SEQ ID NO:43; a sequence encoding the functional truncated GDE polypeptide of SEQ ID NO: 14; and a bGH polyA or pA58 polyA, in particular a pA58 polyA. In a particular embodiment, the expression cassette comprises in the 5′ to 3′ orientation: a CMV promoter such as a mini-CMV promoter, in particular the mini-CMV promoter of SEQ ID NO:43; the sequence of SEQ ID NO: 26 or SEQ ID NO: 27 encoding the functional truncated GDE polypeptide of SEQ ID NO: 14; and a bGH polyA or pA58 polyA, in particular a pA58 polyA. In a particular embodiment, the expression cassette comprises in the 5′ to 3′ orientation: the mini-CMV promoter of SEQ ID NO:43; the sequence of SEQ ID NO: 26 or SEQ ID NO: 27 encoding the functional truncated GDE polypeptide of SEQ ID NO: 14; and the pA58 polyA of SEQ ID NO:42.

In a further embodiment, the expression cassette comprises in the 5′ to 3′ orientation: a CMV promoter such as a mini-CMV promoter, in particular the mini-CMV promoter of SEQ ID NO:43; a sequence encoding the functional truncated GDE polypeptide of SEQ ID NO: 15; and a bGH polyA or pA58 polyA, in particular a pA58 polyA. In a particular embodiment, the expression cassette comprises in the 5′ to 3′ orientation: a CMV promoter such as a mini-CMV promoter, in particular the mini-CMV promoter of SEQ ID NO:43; the sequence of SEQ ID NO: 28 or SEQ ID NO: 29 encoding the functional truncated GDE polypeptide of SEQ ID NO: 15; and a bGH polyA or pA58 polyA, in particular a pA58 polyA. In a particular embodiment, the expression cassette comprises in the 5′ to 3′ orientation: the mini-CMV promoter of SEQ ID NO:43; the sequence of SEQ ID NO: 28 or SEQ ID NO: 29 encoding the functional truncated GDE polypeptide of SEQ ID NO: 15; and the pA58 polyA of SEQ ID NO:42.

In a further embodiment, the expression cassette comprises in the 5′ to 3′ orientation: a CMV promoter such as a mini-CMV promoter, in particular the mini-CMV promoter of SEQ ID NO:43; a sequence encoding the functional truncated GDE polypeptide of SEQ ID NO: 16; and a bGH polyA or pA58 polyA, in particular a pA58 polyA. In a particular embodiment, the expression cassette comprises in the 5′ to 3′ orientation: a CMV promoter such as a mini-CMV promoter, in particular the mini-CMV promoter of SEQ ID NO:43; the sequence of SEQ ID NO: 30 or SEQ ID NO: 31 encoding the functional truncated GDE polypeptide of SEQ ID NO: 16; and a bGH polyA or pA58 polyA, in particular a pA58 polyA. In a particular embodiment, the expression cassette comprises in the 5′ to 3′ orientation: the mini-CMV promoter of SEQ ID NO:43; the sequence of SEQ ID NO: 30 or SEQ ID NO: 31 encoding the functional truncated GDE polypeptide of SEQ ID NO: 16; and the pA58 polyA of SEQ ID NO:42.

In a particular embodiment, the expression cassette comprises or consists of the sequence as shown in SEQ ID NO: 44 to SEQ ID NO:48. In a further embodiment, the expression cassette comprises or consists of a sequence having at least 80% sequence identity, such as at least 85% sequence identity, in particular at least 90%, more particularly at least 91%, 92%, 93%, 94%, 95%, 96%, 97%, 98% or even at least 99% sequence identity to SEQ ID NO: 44 to SEQ ID NO:48.

In a particular embodiment, the expression cassette comprises or consists of the sequence as shown in SEQ ID NO: 44. In a further embodiment, the expression cassette comprises or consists of a sequence having at least 80% sequence identity, such as at least 85% sequence identity, in particular at least 90%, more particularly at least 91%, 92%, 93%, 94%, 95%, 96%, 97%, 98% or even at least 99% sequence identity to SEQ ID NO: 44.

In a preferred embodiment, the expression cassette comprises or consists of the sequence as shown in SEQ ID NO: 45. In a further embodiment, the expression cassette comprises or consists of a sequence having at least 80% sequence identity, such as at least 85% sequence identity, in particular at least 90%, more particularly at least 91%, 92%, 93%, 94%, 95%, 96%, 97%, 98% or even at least 99% sequence identity to SEQ ID NO: 45.

In a further embodiment, the expression cassette comprises or consists of the sequence as shown in SEQ ID NO: 46. In a further embodiment, the expression cassette comprises or consists of a sequence having at least 80% sequence identity, such as at least 85% sequence identity, in particular at least 90%, more particularly at least 91%, 92%, 93%, 94%, 95%, 96%, 97%, 98% or even at least 99% sequence identity to SEQ ID NO: 46.

In a further embodiment, the expression cassette comprises or consists of the sequence as shown in SEQ ID NO: 47. In a further embodiment, the expression cassette comprises or consists of a sequence having at least 80% sequence identity, such as at least 85% sequence identity, in particular at least 90%, more particularly at least 91%, 92%, 93%, 94%, 95%, 96%, 97%, 98% or even at least 99% sequence identity to SEQ ID NO: 47.

In a further embodiment, the expression cassette comprises or consists of the sequence as shown in SEQ ID NO: 48. In a further embodiment, the expression cassette comprises or consists of a sequence having at least 80% sequence identity, such as at least 85% sequence identity, in particular at least 90%, more particularly at least 91%, 92%, 93%, 94%, 95%, 96%, 97%, 98% or even at least 99% sequence identity to SEQ ID NO: 48.

In designing the nucleic acid construct of the invention, one skilled in the art will take care of respecting the size limit of the vector used for delivering said construct to a cell or organ. In particular, one skilled in the art knows that a major limitation of AAV vector is its cargo capacity which may vary from one AAV serotype to another but is thought to be limited to around the size of parental viral genome. For example, 5 kb, is the maximum size usually thought to be packaged into an AAV8 capsid (Wu Z. et al., Mol Ther., 2010, 18 (1): 80-86; Lai Y. et al., Mol Ther., 2010, 18 (1): 75-79; Wang Y. et al., Hum Gene Ther Methods, 2012, 23 (4): 225-33). In addition, during recombinant AAV production, genomes larger than 5 kb are encapsidated with low efficacy and the resulting AAV may contain fragmented genomes reducing the efficacy of gene transfer. Accordingly, those skilled in the art will take care in practicing the present invention to select the components of the nucleic acid construct of the invention so that the resulting nucleic acid sequence, including sequences coding AAV 5′- and 3′-ITRs to preferably not exceed 110% of the cargo capacity of the AAV vector implemented, in particular to preferably not exceed 5 kb. AAV vectors having larger cargo capacity can also be used in the context on the present invention. For example AAV particles lacking Vp2 subunit are shown to successfully package larger genomes (i.e. 6 kb) while preserving integrity of encapsidated genomes (Grieger et al., 2005, J Virol., 79 (15): 9933-9944).

### 4-Vector

The present invention also relates to a vector comprising a nucleic acid molecule or construct as disclosed herein. In a particular embodiment, the vector comprises a nucleic acid molecule or construct encoding a functional truncated GDE polypeptide as defined above.

In particular, the vector of the invention is a vector suitable for protein expression, preferably for use in gene therapy. In one embodiment, the vector is a plasmid vector. In another embodiment, the vector is a nanoparticle containing a nucleic acid molecule of the invention, in particular a messenger RNA encoding the functional truncated GDE polypeptide of the invention. In another embodiment, the vector is a system based on transposons, allowing integration of the nucleic acid molecule or construct of the invention in the genome of the target cell, such as the hyperactive Sleeping Beauty (SB100×) transposon system (Mates et al. 2009). In another embodiment, the vector is a viral vector suitable for gene therapy, targeting any cell of interest such as liver tissue or cells, muscle cell, CNS cells (such as brain cells), or hematopoietic stem cells such as cells of the erythroid lineage (such as erythrocytes). In this case, the nucleic acid construct of the invention also contains sequences suitable for producing an efficient viral vector, as is well known in the art.

Viral vectors are preferred for delivering the nucleic acid molecule or construct of the invention, such as a retroviral vector, for example a lentiviral vector, or a non-pathogenic parvovirus, more preferably an AAV vector. The human parvovirus Adeno-Associated Virus (AAV) is a dependovirus that is naturally defective for replication which is able to integrate into the genome of the infected cell to establish a latent infection. The last property appears to be unique among mammalian viruses because the integration occurs at a specific site in the human genome, called AAVS1, located on chromosome 19 (19q13.3-qter).

Therefore, AAV vectors have arisen considerable interest as potential vectors for human gene therapy. Among the favorable properties of the virus are its lack of association with any human disease, its ability to infect both dividing and non-dividing cells, and the wide range of cell lines derived from different tissues that can be infected.

Among the serotypes of AAVs isolated from human or non-human primates (NHP) and well characterized, human serotype 2 is the first AAV that was developed as a gene transfer vector. Other currently used AAV serotypes include AAV-1, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods.),-3 and AAV-3 variants (such as the AAV3-ST variant comprising an engineered AAV3 capsid with two amino acid changes, S663V+T492V, disclosed in Vercauteren et al., 2016, Mol. Ther. Vol. 24 (6), p. 1042),-3B and AAV-3B variants,-4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p.16026), -7, -8, -9, -2G9, -10 such as cyl0 and -rh10,-rh74,-dj, Anc80, LK03, AAV2i8, porcine AAV serotypes such as AAVpo4 and AAVpo6, and tyrosine, lysine and serine capsid mutants of the AAV serotypes, etc. In addition, other non-natural engineered variants and chimeric AAV can also be useful. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells.

AAV-based recombinant vectors lacking the Rep protein integrate with low efficacy into the host's genome and are mainly present as stable circular episomes that can persist for years in the target cells.

Alternatively to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

In the context of the present invention, the AAV vector comprises an AAV capsid able to transduce the target cells of interest, i.e. cells of the tolerogenic tissue (for example hepatocytes) and cells of the tissue(s) of therapeutic interest such as muscle cells, CNS cells or cardiac cells. In a particular embodiment, the AAV vector comprises an AAV capsid able to transduce muscle cells or cardiac cells. According to a particular embodiment, the AAV vector is of the AAV-1, -2, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods. [Epub ahead of print]),-3 and AAV-3 variants (such as the AAV3-ST variant comprising an engineered AAV3 capsid with two amino acid changes, S663V+T492V, disclosed in Vercauteren et al., 2016, Mol. Ther. Vol. 24 (6), p. 1042),-3B and AAV-3B variants,-4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p.16026),-7, -8, -9, -9P1, -2G9, -10 such as -cyl0 and -rh10, -rh39,-rh43,-rh74,-dj, Anc80, LK03, AAV.PHP, AAV2i8, porcine AAV such as AAVpol (as described in WO2021/219762), AAVpo4 and AAVpo6, and tyrosine, lysine and serine capsid mutants of AAV serotypes. In a particular embodiment, the AAV vector is of the AAV6, AAV8, AAV9, AAV9P1, AAVrh74 or AAV218 serotype (i.e. the AAV vector has a capsid of the AAV6, AAV8, AAV9, AAV9P1, AAVrh74 or AAV218 serotype). In a further particular embodiment, the AAV vector is a pseudotyped vector, i.e. its genome and capsid are derived from AAVs of different serotypes. For example, the pseudotyped AAV vector may be a vector whose genome is derived from one of the above mentioned AAV serotypes, and whose capsid is derived from another serotype. For example, the genome of the pseudotyped vector may have a capsid derived from the AAV6, AAV8, AAV9, AAV9P1, AAVrh74 or AAV218 serotype, and its genome may be derived from and different serotype. In a particular embodiment, the AAV vector has a capsid of the AAV6, AAV8, AAV9 or AAVrh74 serotype, in particular of the AAV6, AAV8, AAV9, or AAV9P1 serotype, more particularly of the AAV6, AAV9 or AAV9P1 serotype.

In a specific embodiment, wherein the vector is for use in delivering the therapeutic transgene to muscle cells, the AAV vector may be selected, among others, in the group consisting of AAV8, AAV9 and AAVrh74.

In another specific embodiment, wherein the vector is for use in delivering the transgene to liver cells, the AAV vector may be selected, among others, in the group consisting of AAV1, AAV5, AAV8, AAV9, AAVrh10, AAVrh39, AAVrh43, AAVrh74, AAV-LK03, AAV2G9, AAV.PHP, AAV-Anc80 and AAV3B.

In a further specific embodiment, wherein the vector is for use in delivering the transgene to the CNS, the AAV vector may be selected, among others, in the group consisting of AAV9, AAV9P1, AAV10 and AAV2G9.

In another embodiment, the capsid is a modified capsid. In the context of the present invention, a “modified capsid” may be a chimeric capsid or capsid comprising one or more variant VP capsid proteins derived from one or more wild-type AAV VP capsid proteins.

In a particular embodiment, the AAV vector is a chimeric vector, i.e. its capsid comprises VP capsid proteins derived from at least two different AAV serotypes, or comprises at least one chimeric VP protein combining VP protein regions or domains derived from at least two AAV serotypes. Examples of such chimeric AAV vectors useful to transduce liver cells are described in Shen et al., Molecular Therapy, 2007 and in Tenney et al., Virology, 2014. For example, a chimeric AAV vector can derive from the combination of an AAV8 capsid sequence with a sequence of an AAV serotype different from the AAV8 serotype, such as any of those specifically mentioned above. In another embodiment, the capsid of the AAV vector comprises one or more variant VP capsid proteins such as those described in WO2015013313, in particular the RHM4-1, RHM15-1, RHM15-2, RHM15-3/RHM15-5, RHM15-4 and RHM15-6 capsid variants, which present a high liver tropism.

In another embodiment, the modified capsid can be derived from capsid modifications inserted by error prone PCR and/or peptide insertion (e.g. as described in Bartel et al., 2011). In a particular embodiment, the capsid includes the P1 insertion, as described in WO2019/193119 or in WO2020/200499, or in WO2022/053630. In addition, capsid variants may include single amino acid changes such as tyrosine mutants (e.g. as described in Zhong et al., 2008). In a particular embodiment, the vector is an AAV9rh74 comprising the P1 insertion (e.g. as described in Sellier, P et al. “Muscle-specific, liver-detargeted adeno-associated virus gene therapy rescues Pompe phenotype in adult and neonate Gaa^{-/-} mice.” Journal of inherited metabolic disease, 10.1002/jimd.12625. 19 May. 2023). In another particular embodiment, the vector is the P1-displaying AAV9 mutant called “AAVMYO” as described in Weinmann et al. (Weinmann, Jonas et al. “Identification of a myotropic AAV by massively parallel in vivo evaluation of barcoded capsid variants.” Nature communications vol. 11,1 5432. 28 Oct. 2020).

In a particular embodiment, the AAV vector is an AAV vector as described in WO2019/193119 or an AAV vector as described in WO2020/200499.

In a further embodiment, the AAV vector is an AAV vector as described in WO2020/216861 or an AAV vector as described in WO2022/003211. In particular, the AAV vector may have a hybrid capsid as described as described in WO2020/216861 or WO2022/003211, such as a hybrid capsid between AAV8 and AAV2/13.

In addition, the genome of the AAV vector may either be a single stranded or self-complementary double-stranded genome (McCarty et al., Gene Therapy, 2003). Self-complementary double-stranded AAV vectors are generated by deleting the terminal resolution site from one of the AAV terminal repeats. These modified vectors, whose replicating genome is half the length of the wild type AAV genome have the tendency to package DNA dimers. In a preferred embodiment, the AAV vector implemented in the practice of the present invention has a single stranded genome, and further preferably comprises an AAV8, AAV9, AAVrh74, AAVrh74-P1, AAV9rh74-P1, AAV218 capsid, or a hybrid capsid as described in WO2020/216861 or WO2022/003211, in particular an AAV8, AAV9, AAV9rh74-P1 or a hybrid capsid as described in WO2020/216861 or WO2022/003211, more particularly an AAV9 capsid.

The AAV vector used for packaging the GDE sequence of the invention can also be modified in order to increase its cargo capacity. For example, AAV vectors lacking Vp2 subunit are shown to successfully package larger genomes (i.e. 6 kb) while preserving integrity of encapsidated genomes (Grieger et al., 2005).

As is known in the art, additional suitable sequences may be introduced in the nucleic acid construct of the invention for obtaining a functional viral vector. Suitable sequences include AAV ITRs.

In a particular embodiment, the AAV vector comprises a muscle-specific promoter as described above, in particular a muscle-specific promoter that presents some leakage of expression into liver cells.

In another particular embodiment of the invention, the AAV vector comprises a liver-specific promoter as described above. The protolerogenic and metabolic properties of the liver are advantageously implemented thanks to this embodiment to develop highly efficient and optimized vectors to express GDE in hepatocytes and to induce immune tolerance to the protein.

In a particular embodiment, a dual recombinant AAV vector system comprising two AAV vectors as described in WO 2018/162748 is used for delivering the nucleic acid molecule or construct encoding the functional truncated GDE polypeptide as defined above. In particular, the dual AAV vector system comprises:
a first AAV vector comprising, between 5′ and 3′ AAV ITRs, a first nucleic acid sequence that encodes a N-terminal part of the truncated GDE polypeptide as defined above, and
a second AAV vector comprising, between 5′ and 3′ AAV ITRs, a second nucleic acid sequence that encodes a part of the truncated GDE polypeptide as defined above, and wherein the first and second nucleic acid sequences encoding said GDE comprise a polynucleotide region that permits the production of a full-length truncated GDE polypeptide as defined above.

In another particular embodiment, the AAV vector is a single AAV vector comprising a nucleic acid molecule or construct encoding a functional truncated GDE polypeptide as defined above.

### 5-Cell

The invention also relates to a cell, in particular an isolated cell, for example a liver cell, a cardiac cell, a CNS cell or a muscle cell, that is transformed or transduced with the nucleic acid molecule, the construct or the vector of the invention. In a particular embodiment, the cell is an isolated human cell. In a further particular embodiment, the cell is not a human embryonic stem cell. The cell of the invention expresses a functional truncated GDE polypeptide as described above. Cells of the invention may be delivered to the subject in need thereof, such as GDE-deficient patient, by any appropriate administration route such as via injection in the liver, in the CNS, in the heart, in the muscle(s) or in the bloodstream of said subject. In a particular embodiment, the invention involves transducing liver or muscle cells, in particular liver or muscle cells of the subject to be treated, and administering said transduced liver and/or muscle cells into which the nucleic acid has been introduced to the subject. In a particular embodiment, the liver cells are liver cells from the patient to be treated, or are liver stem cells that are further transformed, and differentiated in vitro into liver cells, for subsequent administration to the patient. In another embodiment, the cell is a muscle cell from the patient to be treated, or is a muscle stem cell that is further transformed, and optionally differentiated in vitro into muscle cells, for subsequent administration to the patient.

### 6-Pharmaceutical Composition

The present invention also provides pharmaceutical compositions comprising the nucleic acid molecule, the nucleic acid construct, the vector, the functional truncated GDE polypeptide, or the cell of the invention. Such compositions may comprise a therapeutically effective amount of the therapeutic (the nucleic acid molecule, the nucleic acid construct, the vector, the functional truncated GDE polypeptide or the cell of the invention), and a pharmaceutically acceptable carrier. In a specific embodiment, the term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject. In a particular embodiment, the nucleic acid, vector or cell of the invention is formulated in a composition comprising phosphate-buffered saline and supplemented with 0.25% human serum albumin. In another particular embodiment, the nucleic acid, vector or cell of the invention is formulated in a composition comprising ringer lactate and a non-ionic surfactant, such as pluronic F68 at a final concentration of 0.01-0.0001%, such as at a concentration of 0.001%, by weight of the total composition. The formulation may further comprise serum albumin, in particular human serum albumin, such as human serum albumin at 0.25%. Other appropriate formulations for either storage or administration are known in the art, in particular from WO 2005/118792 or Allay et al., 2011.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to, ease pain at the site of the injection.

In an embodiment, the nucleic acid molecule, the nucleic acid construct, the vector, the functional truncated GDE polypeptide or the cell of the invention can be delivered in a vesicle, in particular a liposome. In yet another embodiment, the nucleic acid molecule, the nucleic acid construct, the vector, the functional truncated GDE polypeptide or the cell of the invention can be delivered in a controlled release system.

In a particular embodiment, the nucleic acid molecule is delivered as a mRNA, corresponding to the transcript encoding the functional truncated GDE polypeptide of the invention. In particular, the mRNA of the invention may be delivered using liposomes such as lipid nanoparticle (LNP).

Methods of administration of the nucleic acid molecule, the nucleic acid construct, the vector, the functional truncated polypeptide or the cell of the invention include but are not limited to intradermal, intramuscular, intraperitoneal, intravenous, subcutaneous, intranasal, epidural, and oral routes. In a particular embodiment, the administration is via the intravenous or intramuscular route. The nucleic acid molecule, the nucleic acid construct, the vector, the functional truncated GDE polypeptide or the cell of the invention, whether vectorized or not, may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment, e.g. the liver or the muscle. This may be achieved, for example, by means of an implant, said implant being of a porous, nonporous, or gelatinous material, including membranes, such as sialastic membranes, or fibers.

In a particular embodiment, the functional truncated GDE polypeptide of the invention is used in enzyme replacement therapy (ERT), in particular for treating GSDIII. The term “enzyme replacement therapy” or “ERT” generally refers to the introduction of a purified enzyme into an individual having a deficiency in such enzyme. The administered polypeptide of the invention can be obtained from natural sources, by recombinant expression, produced in vitro, or purified from isolated tissue or fluid. In particular, when used in ERT, the polypeptide of the invention may be administered parenterally, such as via intraperitoneal, intramuscular, intravascular (i.e. intravenous or intraarterial) administration. In particular the polypeptide is administered by intravenous injection. Said administration may be repeated frequently, such as every day, every week, every two weeks or every month, in particular every week or every two weeks.

The amount of the therapeutic (i.e. the nucleic acid molecule, the nucleic acid construct, the vector, the functional truncated GDE polypeptide or the cell of the invention) of the invention which will be effective in the treatment of GSDIII can be determined by standard clinical techniques. In addition, in vivo and/or in vitro assays may optionally be employed to help predict optimal dosage ranges. The precise dose to be employed in the formulation will also depend on the route of administration, and the seriousness of the disease, and should be decided according to the judgment of the practitioner and each patient's circumstances. The dosage of the nucleic acid molecule, the nucleic acid construct, the vector, the functional truncated GDE polypeptide or the cell of the invention administered to the subject in need thereof will vary based on several factors including, without limitation, the route of administration, the specific disease treated, the subject's age or the level of expression necessary to achieve the therapeutic effect. One skilled in the art can readily determine, based on its knowledge in this field, the dosage range required based on these factors and others. In case of a treatment comprising administering a viral vector, such as an AAV vector, to the subject, typical doses of the vector are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×10¹² vg/kg at least 1×10¹³ vg/kg, or at least 1×10¹⁴ vg/kg.

### 7-Method of Treatment

The invention also relates to a method for treating GSDIII, which comprises a step of delivering a therapeutic effective amount of the nucleic acid molecule, the nucleic acid construct, the vector, the functional truncated GDE polypeptide, the pharmaceutical composition or the cell of the invention to a subject in need thereof.

Cirrhosis and hepatocellular carcinoma can also develop in patients with GSDIII. Thus, the invention also relates to a method for treating cirrhosis and hepatocellular carcinoma in a GSDIII patient which comprises a step of delivering a therapeutic effective amount of the nucleic acid molecule, the nucleic acid construct, the vector, the functional truncated GDE polypeptide, the pharmaceutical composition or the cell of the invention to a subject in need thereof.

The invention also relates to a method for treating GSDIII, said method inducing no immune response to the transgene (i.e. to the functional truncated GDE polypeptide encoded by the nucleic acid molecule), or inducing a reduced immune response to the transgene, comprising a step of delivering a therapeutic effective amount of the nucleic acid, the vector, the functional truncated GDE polypeptide, the pharmaceutical composition or the cell of invention to a subject in need thereof. The invention also relates to a method for treating GSDIII, said method comprising repeated administration of a therapeutic effective amount of the nucleic acid, the vector, the functional truncated GDE polypeptide, the pharmaceutical composition or the cell of the invention to a subject in need thereof. In this aspect, the nucleic acid molecule, the nucleic acid construct or the vector of the invention comprises a promoter which is functional in liver cells, thereby allowing immune tolerance to the expressed functional truncated GDE polypeptide produced therefrom. As well, in this aspect, the pharmaceutical composition used in this aspect comprises a nucleic acid molecule, a nucleic acid construct or a vector comprising a promoter which is functional in liver cells. In case of delivery of cells, in particular of liver, cardiac, CNS or muscle cells, said cells may be cells previously collected from the subject in need of the treatment and that were engineered by introducing therein the nucleic acid molecule, the nucleic acid construct or the vector of the invention to thereby make them able to produce the functional truncated GDE polypeptide. According to an embodiment, in the aspect comprising a repeated administration, said administration may be repeated at least once or more, and may even be considered to be done according to a periodic schedule, such as once per week, per month or per year. The periodic schedule may also comprise an administration once every 2, 3, 4, 5, 6, 7, 8, 9 or 10 year, or more than 10 years. In another particular embodiment, administration of each administration of a viral vector of the invention is done using a different virus for each successive administration, thereby avoiding a reduction of efficacy because of a possible immune response against a previously administered viral vector. For example, a first administration of an AAV vector comprising an AAV8 capsid may be done, followed by the administration of a vector comprising an AAV9 capsid.

According to the present invention, a treatment may include curative, alleviation or prophylactic effects. Accordingly, therapeutic and prophylactic treatment includes amelioration of the symptoms of GSDIII or preventing or otherwise reducing the risk of developing a particular glycogen storage disease. The term “prophylactic” may be considered as reducing the severity or the onset of a particular condition. “Prophylactic” also includes preventing reoccurrence of a particular condition in a patient previously diagnosed with the condition. “Therapeutic” may also reduce the severity of an existing condition. The term “treatment” is used herein to refer to any regimen that can benefit an animal, in particular a mammal, more particularly a human subject.

The invention also relates to an ex vivo gene therapy method for the treatment of GSDIII, comprising introducing the nucleic acid molecule, the nucleic acid construct or the vector of the invention into an isolated cell of a patient in need thereof, for example an isolated hematopoietic stem cell, and introducing said cell into said patient in need thereof.

The invention also relates to the nucleic acid molecule, the nucleic acid construct, the vector, the functional truncated GDE polypeptide, the cell or the pharmaceutical composition of the invention for use as a medicament.

The invention also relates to the nucleic acid molecule, the nucleic acid construct, the vector, the functional truncated GDE polypeptide, the cell or the pharmaceutical composition of the invention, for use in a method for treating a disease caused by a mutation in the AGL gene encoding GDE, in particular in a method for treating GSDIII (Cori disease), such as GSDIIIa and GSDIIIb, in particular GSDIIIa.

The invention further relates to the use of the nucleic acid molecule, the nucleic acid construct, the vector, the functional truncated GDE polypeptide, the cell or the pharmaceutical composition of the invention, in the manufacture of a medicament useful for treating GSDIII (Cori disease).

The invention also relates to the nucleic acid molecule, the nucleic acid construct, the vector, the functional truncated GDE polypeptide, the cell or the pharmaceutical composition of the invention, for use in a method for delivering the GDE protein in the affected tissues, in particular in muscle and liver tissues, in particular in muscles.

### EXAMPLES

The invention is further described in detail by reference to the following experimental examples and the attached figures. These examples are provided for purposes of illustration only, and are not intended to be limiting.

In patent applications WO2020/030661 and PCT/EP2021/073309, it was shown that it is possible to generate a truncated form of the GDE protein while retaining enzymatic activity. In particular, the truncated proteins “Δ1b2” and “Δ1b3” were shown to efficiently reduce the amount of glycogen in different muscle tissues. “Δ1b2” corresponds to a GDE polypeptide of SEQ ID NO:1 wherein amino acids 2-81 are deleted. “Δ1b3” corresponds to a GDE polypeptide of SEQ ID NO: 1 wherein amino acids 2-103 are deleted. However, both protein were shown to be less expressed, or less stable than the full size-GDE in vivo. Therefore, the aim was to modify the N-terminal truncation site in order to find a truncated protein that is more stable and better expressed, and ultimately increase the capacity of the protein to reduce glycogen in vivo.

Five N-terminal truncated GDE proteins were designed, as described in the following table:

| | Position of the deleted | |
|---|---|---|
| | amino acids with respect | |
| | to SEQ ID NO: 1 | SEQ ID NO: |
| Δ1b4 | 2-88 | SEQ ID NO: 12 |
| Δ1b5 | 2-99 | SEQ ID NO: 13 |
| Δ1b6 | 2-111 | SEQ ID NO: 14 |
| Δ1b7 | 2-115 | SEQ ID NO: 15 |
| Δ1b8 | 2-123 | SEQ ID NO: 16 |

To evaluate the activity of the truncated GDEs, AAV vectors expressing the truncated GDEs (Δ1b5 or Δ1b3) or the full-size protein (AAV-GDE-full size) were produced. AAV9rh74-P1 vectors (i.e. AAV vectors having a hybrid capsid between AAV9 and AAVrh74 modified with peptide P1) were used throughout the experiments. The transgene was cloned in a transgene expression cassette optimized for muscle expression and composed of a mini CMV promoter and a pA58 poly adenylation signal. The cassette of the Δ1b5, Δ1b3 and GDE-full size constructs is as shown in SEQ ID NO:45, SEQ ID NO:49 and SEQ ID NO:50, respectively.

### Example 1

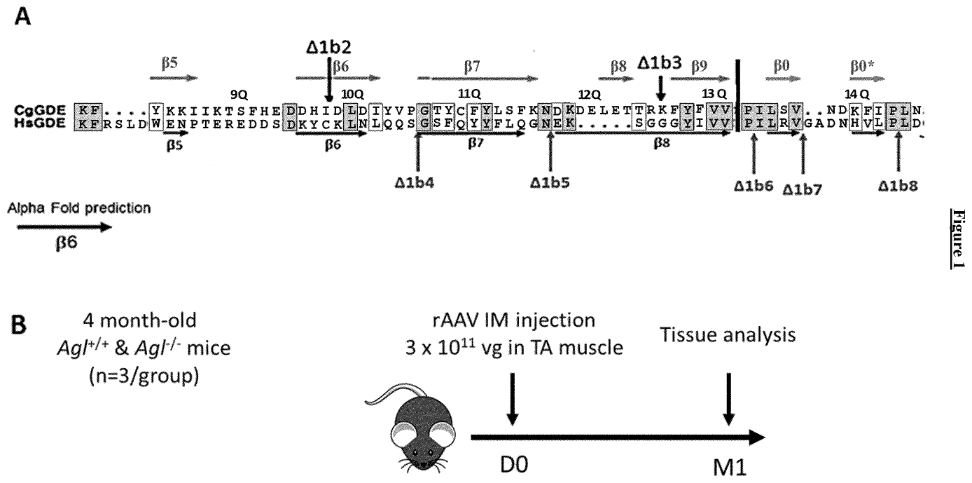

The vectors were injected in 4-6 month-old GSDIII mice, in the left tibialis anterior muscle, at the dose of 3×10¹¹ vg/mouse (FIG. 1). PBS-injected wild-type (WT, PBS) or knockout mice (KO, PBS) were used as controls. Mice were sacrificed 1 month after injection for measuring glycogen content in left tibialis anterior muscle.

### Measurement of Glycogen Content

Glycogen content was measured indirectly in tissue homogenates as the glucose released after total digestion with Aspergillus Niger amyloglucosidase (Sigma-Aldrich, ref Δ1602). Samples were incubated for 10 min at 95° C. and then cooled at 4° C. After the addition of amyloglucosidase (final concentration 4 U/mL) and potassium acetate pH 5.5 (final concentration 25 mM) at 37° C. for 90 min, the reaction was stopped by incubating samples for 10 min at 95° C. A control reaction without amyloglucosidase was prepared for each sample and was incubated in the same conditions. The glucose released was determined using a glucose assay kit (Sigma-Aldrich) and the resulting absorbance was acquired on an EnSpire alpha plate reader (PerkinElmer) at a wavelength of 540 nm. Glucose released after amyloglucosidase was then normalized by the total protein concentration.

### Measurement of GDE Expression

Mouse tissues were homogenized in phosphate buffer saline (PBS, ThermoFisher Scientific) with complete™ protease inhibitor cocktail (Roche, ref 4693132001). Protein concentration was determined using the Pierce™ BCA Protein Assay (Thermo Fisher Scientific) according to the manufacturer's instructions. A fraction of 50 μg of total proteins were loaded in each well for both PBS- and rAAV-injected Agl^{-/-} mice and 10 μg of total proteins per well for PBS-injected Agl^{+/+} mice. SDS-PAGE electrophoresis was performed in a 4-12% Bis-Tris gradient polyacrylamide gel (NuPAGE™ Invitrogen). After transfer, the membrane was blocked with Intercept Blocking buffer (LI-COR Biosciences) and incubated with an anti-GDE rabbit polyclonal antibody (16582-1-AP, Proteintech) and an anti-vinculin mouse monoclonal antibody (V9131, Sigma). The membrane was washed and incubated with the appropriate secondary antibody (LI-COR Biosciences) and visualized by Odyssey imaging system (LI-COR Biosciences).

### Quantification of Vector Genome Copy Number

DNA was extracted from tissue homogenate using the Nucleomag Pathogen extraction kit from Macherey-Nagel (#744210.4) following the manufacturer instructions. Vector genome copy number was determined using a qPCR assay. The PCR primers used in the reaction were located in the glucosyltransferase domain of the full-length and truncated codon-optimized GDE (SEQ-33; Forward: 5′-CTG AAG CTG TGG GAG TTC TT-3′ (SEQ ID NO:59) and Reverse: 5′-CTC TTG GTC ACT CTT CTG TTC TC-3′ (SEQ ID NO:60)). As an internal control, primers located within the mouse (Forward: 5′-AAA ACG AGC AGT GAC GTG AGC-3′ (SEQ ID NO:61) and Reverse: 5′-TTC AGT CAT GCT GCT AGC GC-3′ (SEQ ID NO:62)) Titin gene were used.

### Results

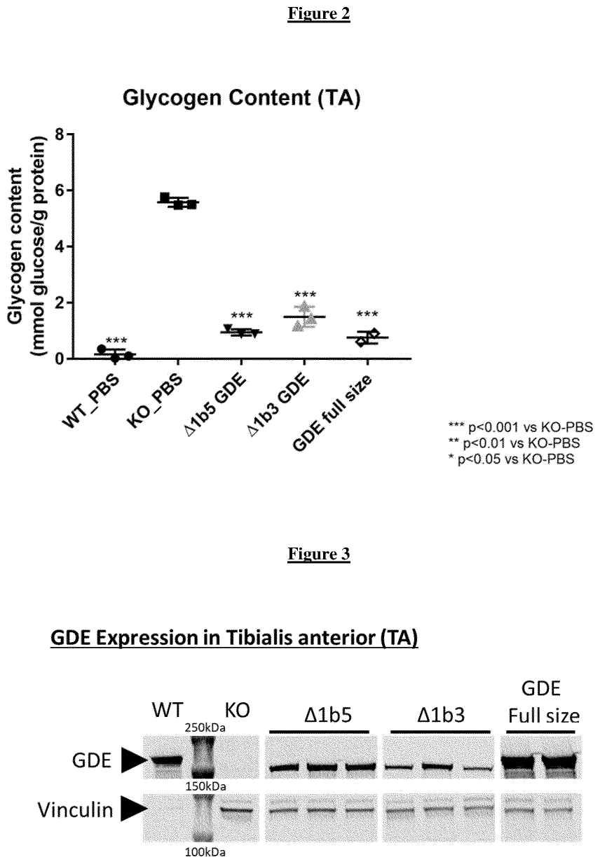

The results show a significant decrease in glycogen content, for the mice injected with truncated GDE constructs compared to the control group, injected with PBS. The GDE proteins “Δ1b3”, “Δ1b5” and the full-length form of GDE (GDE full size) showed significant efficacy in terms of glycogen depletion in the injected muscle (FIG. 2).

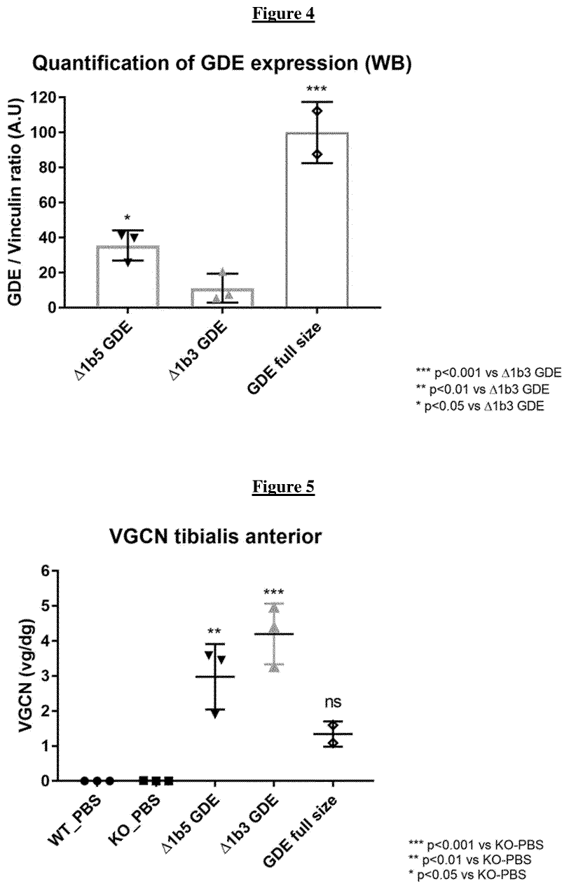

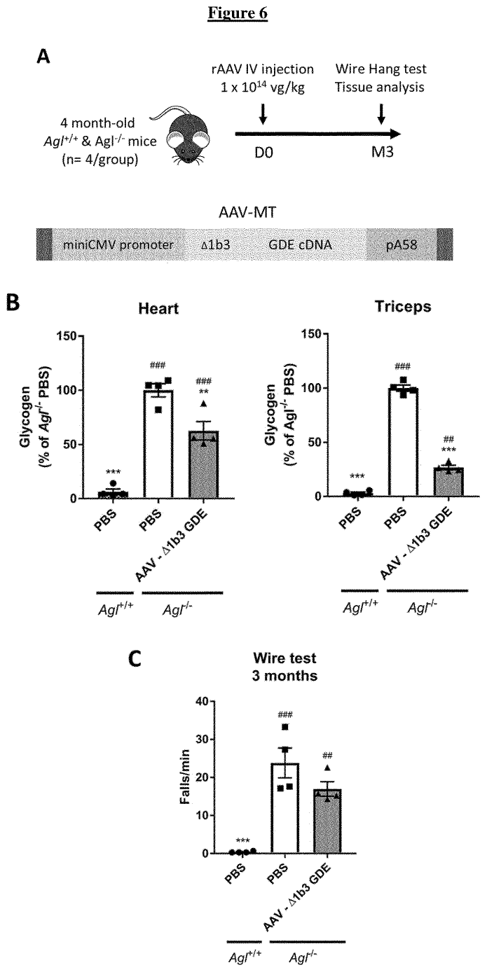

Δ1b5 GDE construct shows a much better protein expression level, when compared to the Δ1b3 GDE construct (FIGS. 3 and 4) even though the truncation sites are very close (FIG. 1). This experiment shows that it is not possible to predict the expression level of a new truncated protein based solely on the position of the truncation site.

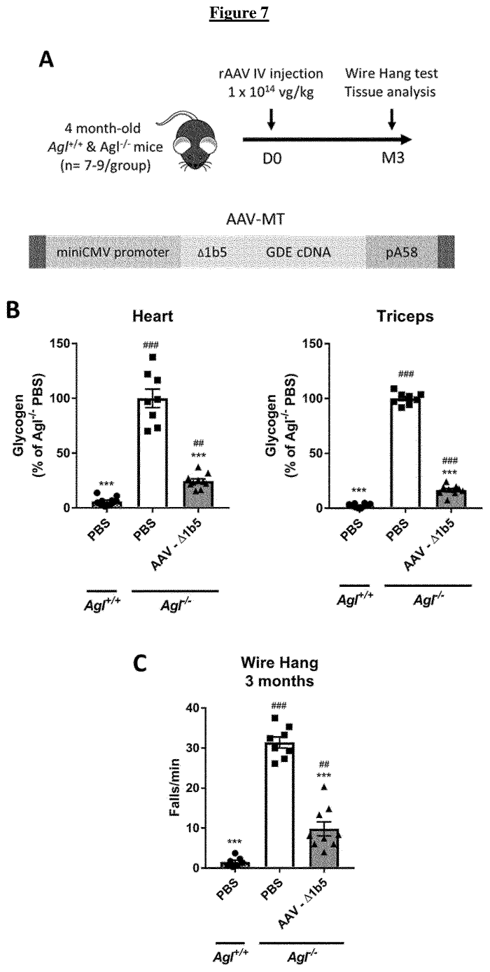

Furthermore, quantification of vector genome copy number (VGCN) in the injected muscle showed lower VGCN in the tibialis anterior injected with the Δ1b5 GDE construct compared to the Δ1b3 GDE construct (FIG. 5), emphasizing that the higher expression of the Δ1b5 GDE construct observed in

FIGS. 3-4 is not due to higher amount of injected rAAV vector.

### Example 2

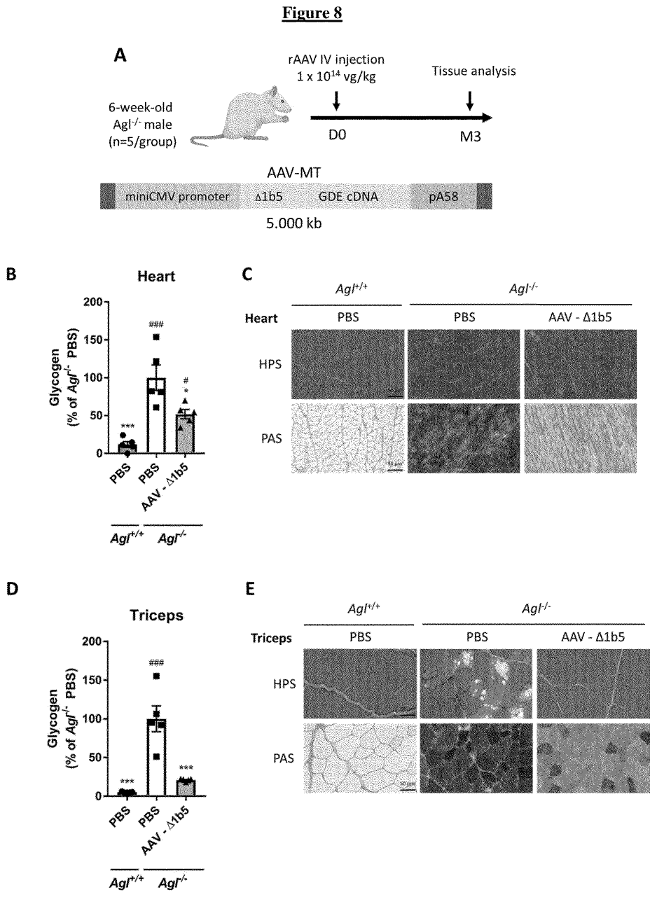

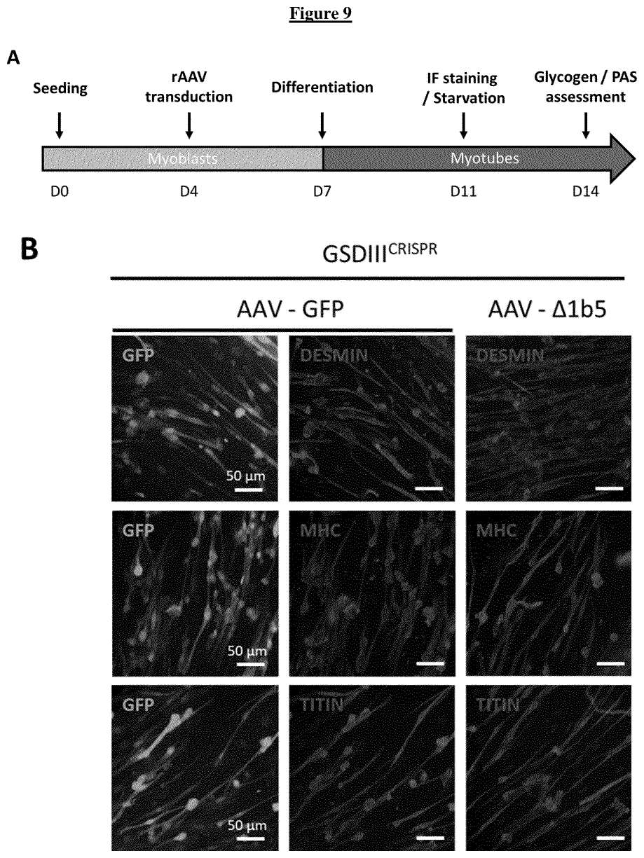

The efficacy of an rAAV vector encoding the Δ1b3-GDE (FIG. 6) or the Δ1b5-GDE (FIG. 7) was evaluated in a mouse model of GSDIII. 4-month-old male Agl^{-/-} mice were injected in the tail vein with an rAAV vector encoding either Δ1b3-GDE (FIG. 6) or Δ1b5-GDE (FIG. 7), at the dose of 1×10¹⁴ vg/kg. PBS-injected Agl^{+/+} and Agl^{-/-} mice were used as controls. Glycogen content was measured in heart and triceps at euthanasia, as described above. Wire-hang test, expressed as number of falls per minute, was performed 3 months after vector injection. A 4-mm-thick wire was used to record the number of falls over a period of 3 min. The average number of falls per minute was reported for each animal.

### Results

The results show a significant decrease in glycogen content in heart and triceps, for the mice injected with truncated Δ1b3-GDE construct (FIG. 6B) and Δ1b5-GDE construct (FIG. 7B) compared to the control group, injected with PBS. The Δlb5-GDE construct showed a better efficacy in terms of glycogen depletion in heart and triceps, when compared to the Δ1b3-GDE construct.

In addition, treatment with rAAV-Δ1b3-GDE and rAAV-Δ1b5-GDE improved muscle strength assessed by wire-hang. Three months post-injection, mice showed less frequency of falls compared to the PBS-injected group (as shown in FIGS. 6C and 7C).

Importantly, treatment with rAAV-Δ1b5-GDE is shown to be much more efficient for rescuing muscle strength than rAAV-Δ1b3-GDE. Three months post-injection rAAV-Δ1b5-GDE injected mice showed less frequency of falls compared to the PBS-injected group (9.8 falls/min±1.8 vs. 31.4 falls/min±1.4, corresponding to a 69% decrease), almost reaching wild-type levels (FIG. 7C). In comparison, rAAV-Δ1b3-GDE injected mice showed around 17 falls/min±1.9 three months post-injection (vs. 23.8±3.9 in PBS injected group, corresponding only to 29% decrease).

These data clearly demonstrate that treatment with rAAV-Δ1b5-GDE reverses the muscle impairment in adult, symptomatic GSDIII mice at both biochemical and functional level.

### Example 3

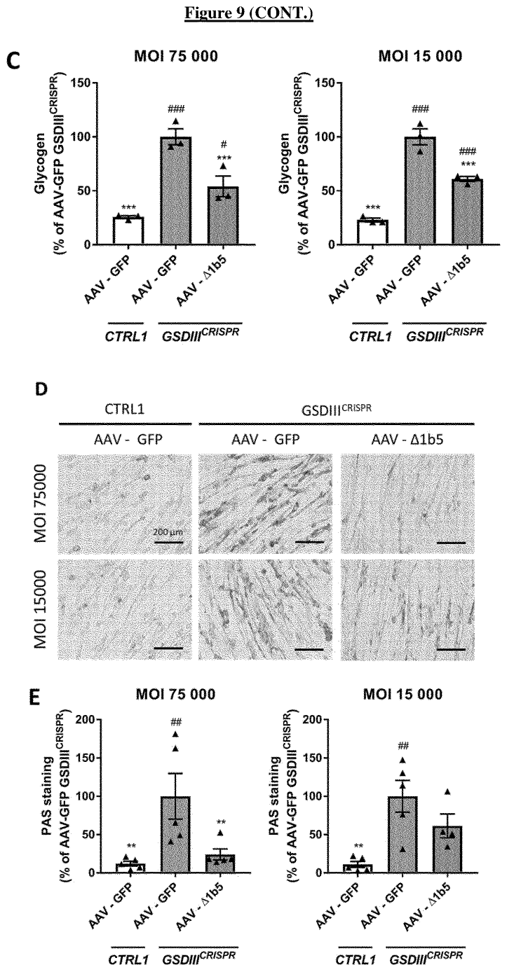

To confirm the efficacy of the rAAV-Δlb5-GDE construct in a larger animal model, the vector was administered to a rat model of GSDIII. 6-week-old Agl^{-/-} rats, treated with 1×10¹⁴ vg/kg of rAAV-Δ1b5-GDE via tail vein injection, were analyzed three months post-injection (FIG. 8A). PBS-injected Agl^{-/-} and Agl^{+/+} rats were used as controls. Glycogen content was measured in heart and triceps at euthanasia, as described above.

Histological analysis of heart and triceps was carried out with HPS (hematoxylin phloxine saffron staining) and PAS (periodic acid-Schiff staining) staining. For muscle histology, heart and triceps were snap-frozen in isopentane previously chilled in liquid nitrogen. Serial 8-μm cross sections were cut in a Leica CM3050 S cryostat (Leica Biosystems). To minimize sampling error, 2 sections of each specimen were obtained and stained with HPS or PAS according to standard procedures. Images were digitalized using Axioscan Z1 slide scanner (Zeiss, Germany) under a Zeiss Plan-Apochromat 10×/0.45 M27 dry objectif (ZEISS, Germany). Tile scan images were reconstructed with ZEN software (ZEISS, Germany).

### Results

A significant, 50% reduction in glycogen accumulation was observed in the heart in rAAV-Δ1b5-GDE injected rats three months post injection (FIG. 8B) that was also noticeable in the PAS staining (FIG. 8C). An almost complete glycogen clearance was also observed in skeletal muscles such as the triceps (FIG. 8D) with normalization of muscle architecture and reduction of glycogen accumulation on histological analysis (FIG. 8E).

In conclusion, the data obtained by the treatment of Agl^{-/-} rats with an rAAV vector encoding for Δ1b5-GDE confirm those obtained in the mouse model of the disease and further support the clinical translation of rAAV-Δ1b5-GDE to treat the muscle disease in GSDIII patients.

### Example 4

The activity of the truncated Δ1b5-GDE was also evaluated in a human pathological context, using an in vitro human skeletal muscle model of GSDIII. Skeletal muscle myoblasts and myotubes were derived from human induced pluripotent stem cells (hiPSCs) edited by CRISPR/Cas9 technology (GSDIII^{CRISPR}) to knock-down the AGL gene.

Transduction of hiPSC-Derived Skeletal Muscle Cells

The GSDIII^{CRISPR} hiPSCs have been previously generated, using CRISPR-knock down of the AGL gene (Rossiaud et al, Pathological modeling of Glycogen Storage Disease type III with CRISPR/Cas9 edited human pluripotent stem cells, Front. Cell Dev. Biol., 11 May 2023). Controls hiPSCs were the isogenic cell line (CTRL1). GSDIII^{CRISPR} and CTRL1 hiPSCs were differentiated into skeletal myoblasts as previously described (Rossiaud et al). After expansion in 96-well plate, hiPSC-derived skeletal myoblasts were transduced with rAAV vectors encoding either GFP or Δlb5-GDE under the control of the miniCMV promoter, at a MOI of either 75 000 or 15 000 for 72 hours. Then, hiPSC-derived skeletal myoblasts were differentiated into myotubes, as previously described (Rossiaud et al).

### Measurement of Glycogen Content in Skeletal Myotubes

After 4 days of differentiation into myotubes, hiPSC-derived skeletal myotubes were starved for 3 days in a no-glucose DMEM medium with 10% fetal bovine serum (ref 11966025, ThermoFisher Scientific) in order to induce glycogen degradation in CTRL1 myotubes as previously described (Rossiaud et al). Cells were lysed using HCl 0.3M and Tris 450 mM pH 8.0. Glycogen was then quantified using the Glycogen-Glo™ assay kit (ref CS1823B01, Promega) and normalized using the CellTiter-Glo™ Luminescent Cell Viability Assay (ref G7570, Promega).

### Immunostaining Assay.

Skeletal myotubes derived from hiPSCs were fixed with 4% paraformaldehyde (Euromedex) for 10 min at room temperature. After two washes in PBS, cells were permeabilized with 0.5% Triton X-100 for 5 min and blocked in PBS solution supplemented with 1% bovine serum albumin (BSA, Sigma) for 1 h at room temperature. Skeletal myotubes were stained for specific skeletal myogenic markers overnight at 4° C. using primary antibodies (Desmin, ref AF3844 R&D 1:200; MF20, ref 3ea DSHB 1:50; Titin ref T5650 US Biological 1:50). After 3 washes in PBS, staining was revealed by appropriate Alexa Fluor secondary antibodies 1:1000 (Donkey anti-goat AF488 ref Δ11055 Invitrogen 1:1000; Donkey anti-mouse AF488 ref Δ21202 Invitrogen 1:1000) in the dark for 1 h at room temperature, and nuclei were visualized with Hoechst solution 1:3000 (Invitrogen). Cell imaging was carried out with a Zen Black software-associated LSM-800 confocal microscope (Zeiss) with a 20× objective.

### Periodic Acid-Schiff (PAS) Staining on Skeletal Myotubes.

PAS staining on hiPSC-derived skeletal myotubes was performed with the PAS Staining Kit (Sigma-Aldrich) following the manufacturer's instructions. Briefly, cells were fixed with 4% paraformaldehyde for 10 min at room temperature. After 2 washes in PBS, cells were treated with periodic acid for 5 minutes at room temperature. After 3 washes in distilled water, cells were treated with Schiff's reagent for 15 min at room temperature. Finally, after 4 washes in tap water, staining was visualized using an EVOS XL Core microscope (Invitrogen). Images were processed and analyzed using FIJI custom-made scripts. First, colors were split and only the green channel was kept as it was the most contrasted one. Then, images were manually thresholded into binary images where PAS signal was black and background white. The threshold was set for maximizing the difference between genotypes, and once calculated, the same threshold was applied to all images to quantify. The quantification of PAS staining was obtained using this formula: Area of PAS staining/Total area of image ×100, giving then a percentage of PAS staining within the image.

### Results

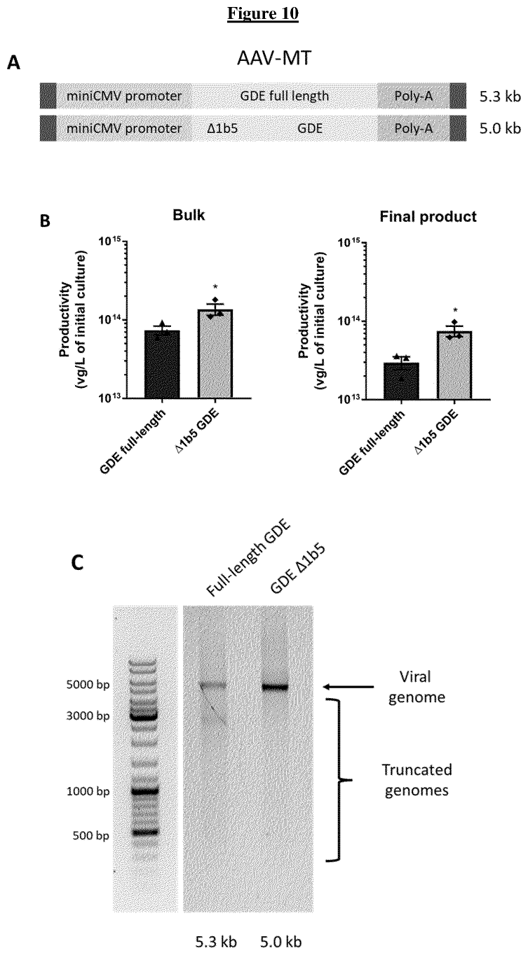

GSDIII^{CRISPR} and CTRL1 hiPSC-derived skeletal myoblasts were transduced with an rAAV vector expressing Δ1b5-GDE or GFP following a protocol previously reported (Rossiaud et al) (FIG. 9A). After transduction with rAAV, skeletal myoblasts were differentiated into skeletal myotubes. Transduction with rAAV expressing GFP or Δ1b5-GDE did not alter the differentiation of myoblasts into myotubes, that showed similar expression of skeletal myogenic markers by immunostaining analysis (FIG. 9B). GSDIII^{CRISPR} skeletal myotubes transduced with rAAV expressing Δ1b5-GDE exhibited a significant reduction of glycogen content when compared to GSDIII^{CRISPR} myotubes transduced with the control vector (FIG. 9C). PAS staining performed on transduced-skeletal myotubes confirmed these results (FIG. 9D-E).

These data demonstrate that the truncated Δ1b5-GDE clears glycogen not only in mouse and rat muscles, but also in a human skeletal muscle model of GSDIII without any specific toxicity.

### Example 5

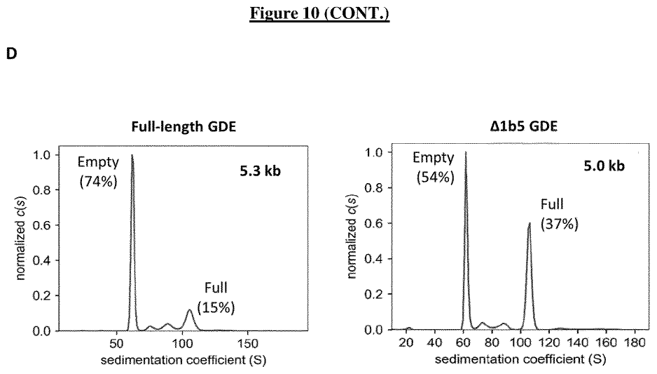

Oversized rAAV vector production has a large impact on the yields and the quality of the final product, which are critical parameters for the translation to the clinic of rAAV-based gene therapies. To evaluate the impact of the use of the truncated Δ1b5-GDE on the production yields and the quality of the vector, an oversized expression cassette expressing human full-length GDE (5.3 kb) was compared to the 5 kb expression cassette encoding the truncated Δ1b5-GDE (FIG. 10). Small scale rAAV productions (50 mL culture) were performed in triplicate for each vector and viral titers were measured both before (bulk) and after purification (final product) for each triplicate. rAAV vector DNA was extracted and loaded on a 1% Agarose gel to assess genome integrity. Analytical ultracentrifugation (AUC) was carried out to analyze the proportion of full and empty particles.

### Viral Genome Analysis on Agarose Gel

DNA was extracted using the High Pure Viral Nucleic Acid Kit (Roche, ref 11858874001). Purified viral DNA was then loaded on a 1%-Agarose gel (Eurobio Scientific) stained with SybrSafe® Gel Stain (Invitrogen) to visualize the viral DNA. Expected genome size range from 5.0 to 5.3 kb.

### Analytical Ultracentrifugation (AUC).

AUC measures the sedimentation coefficient of macromolecules by following over time the optical density of a sample subjected to ultracentrifugation. The difference in the sedimentation coefficient, measured by Raleigh interference or 260-nm absorbance, depends on the content of viral genome in the capsid. AUC analysis was performed using a Proteome Lab XL-I (Beckman Coulter, Indianapolis, IN). An aliquot of 400 μL rAAV vector and 400 μL formulation buffer were loaded into a two-sector velocity cell. Sedimentation velocity centrifugation was performed at 20,000 rpm and 20° C. Absorbance (260 nm) and Raleigh interference optics were used to simultaneously record the radial concentration as a function of time until the lightest sedimenting component cleared the optical window (approximately 1.5 h). Absorbance data required the use of extinction coefficients to calculate the molar concentration and the percent value of the empty and genome-containing capsids. Molar concentrations of both genome-containing and empty capsids were calculated using Beer's law, and percentages of full genome-containing and empty capsids were calculated.

### Results

The use of the Δ1b5-GDE cDNA allowed for approximately ten-fold increase of rAAV production yields (FIG. 10B) while allowing for the efficient encapsidation of a non-truncated genome (FIG. 10C). Improved yields and genome quality resulted in dramatically improved full to empty particles ratios as measured by analytical ultracentrifugation (AUC), with up to 37% of full particles measured in AAV-Δ1b5-GDE, versus only 15% of full particles measured in AAV-full-length-GDE (FIG. 10D). Taken together, these data indicate that while Δ1b5-GDE has an in vivo efficacy similar to the full-size enzyme, it allows to produce rAAV vectors with higher yields and quality thus providing a promising gene therapy candidate for the treatment of GSDIII.

1-15. (canceled)

16. A functional truncated GDE polypeptide, wherein said functional truncated GDE polypeptide comprises a deletion with respect to a reference functional full-length human GDE sequence, and wherein said deletion consists of the deletion of amino acids in the N-terminal part of the reference functional full-length human GDE sequence in such a way that the first six amino acids at the N-terminus of the functional truncated GDE polypeptide are:
(SEQ ID NO: 7)
MGSFQY;
(SEQ ID NO: 8)
MEKSGG;
(SEQ ID NO: 9)
MILRVG;
(SEQ ID NO: 10)
MGADNH;
or
(SEQ ID NO: 11)
MLDCVT.

17. The functional truncated GDE polypeptide of claim 16, wherein the deletion consists of the deletion of amino acids in the N-terminal part of the reference functional full-length human GDE sequence in such a way that the first six amino acids at the N-terminus of the functional truncated GDE polypeptide are MEKSGG (SEQ ID NO:8) or MILRVG (SEQ ID NO: 9).

18. The functional truncated GDE polypeptide of claim 16, wherein the reference functional full-length human GDE has an amino acid sequence as shown in SEQ ID NO: 1, SEQ ID NO:2, SEQ ID NO:3, SEQ ID NO:4, SEQ ID NO:5 or SEQ ID NO:6, or has an amino acid sequence having at least 80, 85, 90, 95, 96, 97, 98 or at least 99 percent sequence identity to SEQ ID NO:1, SEQ ID NO:2, SEQ ID NO:3, SEQ ID NO:4, SEQ ID NO:5 or SEQ ID NO: 6.

19. The functional truncated GDE polypeptide of claim 16, wherein the reference functional full-length human GDE has an amino acid sequence as shown in SEQ ID NO: 1 or SEQ ID NO:4 or has an amino acid sequence having at least 80, 85, 90, 95, 96, 97, 98 or at least 99 percent sequence identity to SEQ ID NO:1 or SEQ ID NO:4.

20. The functional truncated GDE polypeptide of claim 16, wherein the functional truncated GDE polypeptide further comprises a deletion or a combination of deletions with respect to the reference functional full-length human GDE sequence, such as a deletion or a combination of deletions in the C-terminal part of the GDE sequence, or a deletion or a combination of deletions in the central domain of the GDE.

21. The functional truncated GDE polypeptide of claim 20, wherein the functional truncated GDE polypeptide further comprises a deletion or a combination of deletions with respect to SEQ ID NO:1, SEQ ID NO:2, SEQ ID NO:3, SEQ ID NO:4, SEQ ID NO:5 or SEQ ID NO:6, and wherein the deletion(s) is (are) selected from any deletion referred to as Δ1, Δ2, 43, 44, 45, Δ6, and 47 in table 2.

22. The functional truncated GDE polypeptide of claim 16, wherein the functional truncated GDE polypeptide has an amino acid sequence as shown in SEQ ID NOs: 12-16 or has an amino acid sequence having at least 80, 85, 90, 95, 96, 97, 98 or at least 99 percent sequence identity to SEQ ID NOs: 12-16.

23. The functional truncated GDE polypeptide of claim 16, wherein the functional truncated GDE polypeptide has an amino acid sequence as shown in SEQ ID NO:13 or 14 or has an amino acid sequence having at least 80, 85, 90, 95, 96, 97, 98 or at least 99 percent sequence identity to SEQ ID NO: 13 or 14.

24. A nucleic acid molecule encoding the functional truncated GDE polypeptide of claim 16.

25. An expression cassette, comprising:
a promoter;
optionally, an intron;
the nucleic acid molecule of claim 24; and
a polyadenylation signal.

26. A vector comprising the nucleic acid molecule of claim 24 or an expression cassette comprising said nucleic acid molecule, a promoter, a polyadenylation signal and, optionally, an intron.

27. The vector of claim 26, which is an AAV vector.

28. The vector of claim 26, wherein the vector is a viral vector.

29. An isolated cell transformed with the nucleic acid molecule of claim 24, an expression cassette comprising said nucleic acid molecule, a promoter, a polyadenylation signal and, optionally, an intron, or a vector comprising said nucleic acid molecule or expression cassette.

30. The isolated cell of claim 29, wherein the cell is a liver cell, a muscle cell, a cardiac cell or CNS cell.

31. A method of treating a disease caused by a mutation in the AGL gene encoding GDE comprising administering to a subject in need of treatment:
a functional truncated GDE polypeptide of claim 16;
a nucleic acid encoding said functional truncated GDE polypeptide;
an expression cassette comprising said nucleic acid molecule, a promoter, a polyadenylation signal and, optionally, an intron;
a vector comprising said nucleic acid or expression cassette; or
a cell comprising said nucleic acid, an expression cassette comprising said nucleic acid molecule, a promoter, a polyadenylation signal and, optionally, an intron, or a vector comprising said nucleic acid molecule or expression cassette.

32. A method of treating GDSIII (Cori Disease) comprising administering to a subject in need of treatment:
a functional truncated GDE polypeptide of claim 16;
a nucleic acid encoding said functional truncated GDE polypeptide;
an expression cassette comprising said nucleic acid molecule, a promoter, a polyadenylation signal and, optionally, an intron;
a vector comprising said nucleic acid or expression cassette; or
a cell comprising said nucleic acid, an expression cassette comprising said nucleic acid molecule, a promoter, a polyadenylation signal and, optionally, an intron, or a vector comprising said nucleic acid molecule or expression cassette.
